(19) United States
(12) Patent Application Publication
(10) Pub. No.: US 2002/0146734 A1
(43) Pub. Date: Oct. 10, 2002

(54) Method and apparatus for labeling and analyzing cellular components

(75) Inventors: William E. Ortyn, Bainbridge Island, WA (US); David A. Basiji, Seattle, WA (US)

Correspondence Address: LAW OFFICES OF RONALD M. ANDERSON, Suite 507, 600 - 108th Avenue N.E., Bellevue, WA, 98004, US

(73) Assignee: Amnis Corporation

(21) Appl. No.: 10/082,805

(22) Filed: Feb. 21, 2002

Related U.S. Application Data
(60) Provisional application No. 60/270,518, filed on Feb. 21, 2001.

(51) Int. Cl. C12Q 1/68

(52) U.S. Cl. 435/6

(57) ABSTRACT

A labeling method that labels an object or specific features of an object with labeled probes that provide a multiplexed signal that can be analyzed by spectral decomposition. This binary and higher encoding scheme can be employed to label components of biological cells. In each encoding scheme, labeled probes that selectively bind to a specific feature are required. The labeled probes include a binding element that binds to the feature, and at least one signaling component that generates a detectable signal, preferably a spectral signature. In one embodiment, adding multiple fluorescent dye molecules to each binding element provides the multiplexed signal. In another embodiment, adding only one signal compound to each binding element provides the multiplexed signal, such that some of the binding elements have a different signal compound added. The different signal compounds provide the multiplexed signal.

### RELATED APPLICATION

This application is based on a prior co-pending provisional application Serial No. 60/270,518, filed on Feb. 21, 2001, the benefit of the filing date of which is hereby claimed under 35 U.S.C. §119(e).

### FIELD OF THE INVENTION

The present invention generally relates to a method and apparatus employed to probe and simultaneously analyze a plurality of cellular features, and more specifically, employs biomolecular probes labeled with different fluorescent markers in a multiplex color encoding scheme in which each probe produces a unique combination of colors.

### BACKGROUND OF THE INVENTION

The analysis of cells often involves the probing of various cellular components with fluorescent or absorbent substances to determine the presence, absence, abundance, and distribution of the target components within the cell. It is desirable to employ a large number of probes within a cell to facilitate studying the relationships between different cellular components. In some cases, the probes employed include an absorbent or fluorescent substance that exhibits a characteristic specificity for a cellular component, as in the case of certain DNA labels (e.g., ethidium bromide). However, most probes comprise a binding element combined with a signaling element. The binding element binds to a specific cellular component and is chemically linked to the signaling element, which produces a detectable signal—generally a fluorescent emission. Typically, fluorescence detection systems accommodate only a limited number of fluorescent colors based on the available excitation wavelengths and the width of the spectral detection bands. The use of a separate signaling element gives a researcher control over the fluorescent color associated with each probe as a means of optimizing the results within the constraints of an illumination and detection system. Nevertheless, the total number of probes available to the researcher is typically on the order of the number of fluorescent colors that can be detected with a given apparatus. Hence, there exists a need to enable analysis of a large number of cellular probes with only a limited number of fluorescent colors.

A wide variety of probes are available, enabling the analysis of cell type, viability, phase in the cell cycle, the level and activity of numerous biomolecules, as well as other types of information. For example, T-lymphocytes can be identified in whole blood by the binding of an FITC-labeled monoclonal antibody to membrane-bound CD4 proteins. As another example, the viability of cells in a sample can be analyzed by exposing the cells to propidium iodide, which cannot penetrate live cells, but readily labels dead cells. In still another example, a cancerous sub-population of cells can be identified by labeling cellular deoxyribonucleic acid (DNA) with 4′, 6-diamidino-2-phenylindole (DAPI) fluorescent dye and producing a histogram of the DNA content of the population of cells. Deviations of the histogram from that of a normal cell population can indicate the presence of cancerous cells undergoing increased DNA replication and cell division. Depending on the analytical instrumentation employed, multiple probes can be combined within a cell and quantified independently.

Flow cytometry is one technique that allows the use of multiple probes within cells for identifying cellular components. Flow cytometers are non-imaging devices that measure the intensities of multiple fluorescent probes simultaneously. The relative intensity of each of these fluorescent probes is indicative of various conditions within the cells in the sample population. From this information, conclusions may be drawn regarding the disease state of the cell or the reactivity of the cells to various drug candidates, etc. In general, it is desirable to employ as many different probes as possible in order to increase the amount of information obtained from the cells being analyzed. Typical flow cytometers are equipped with four photomultiplier tubes (PMTs), each PMT being dedicated to detecting light of a different fluorescent color. Higher performance instruments may detect six or eight different colors. However, their lack of spatial resolution precludes conventional flow cytometers from determining the spatial origin of fluorescence signals within a cell. Indeed, the fluorescence emission of each color is integrated over the entire cell. Therefore, flow cytometry cannot be used for assays that require imaging, including numerous assays used in drug discovery. Further, the lack of spatial resolution effectively prevents the use of probes labeled with multiple colors, since probes that share colors cannot be distinguished from each other in flow cytometric measurements. Accordingly, the number of probes that can be measured in a flow cytometer is limited to the number of colors that can be detected. Hence, there is clearly a need to enable imaging of multiple fluorescent colors at high throughput for the purposes of implementing quantitative image-based assays and enabling the use of multiplexed color encoding of probes.

In microscopy applications, cells are fixed to a slide and imaged onto a pixilated detector or scanned with a confocal laser arrangement. Three-color imagery is common for direct human observation, but most analytical microscopy platforms image one color at a time. Methods have been developed to serially image cells and cellular components in different colors, using the spatial resolution of microscopy to increase the number of probes that can be detected.

One prior art use of microscopy for the detection of large numbers of probes is described in Prenatal Diagnosis 18:1181-1185 (1998) entitled “Poly-Fish: A Technique of Repeated Hybridizations That Improves Cytogenetic Analysis of Fetal Cells in Maternal Blood.” The article discloses a method for multicolor fluorescence in situ hybridization (FISH) labeling of chromosomes and other cellular components in which the number of chromosomes may be directly counted to determine the incidence of trisomy, a task that requires both spatial resolution and multiple probes. In this process, three separate probes are simultaneously hybridized to three different chromosomes in interphase nuclei and then analyzed on a fluorescence microscope with a triple bandpass filter. The locations of interesting nuclei are noted, the first probes stripped from the slide, followed by re-hybridization using new probes linked to the same set of three colors to enumerate a different set of chromosomes. In all, nine separate hybridization rounds are performed in which each hybridization involves up to three probes and three colors. As disclosed in the above-referenced article, Chromosomes X, Y, 1, 5, 6, 13, 18, and 21 were enumerated, with some chromosomes being probed multiple times. The article concludes that using this multi-step probing protocol, all 24 human chromosomes could potentially be analyzed using only three colors. However, the procedure is difficult to automate due to the complex probing protocol, the protocol is error prone due to incomplete denaturation of probes between hybridizations, and throughput is low due to serial hybridization steps and the need to produce multiple images of each cell. Many of these drawbacks could be addressed in a single-step process employing all of the required probes simultaneously.

U.S. Pat. No. 6,066,459 discloses a method by which all chromosomes are uniquely identified in a single-step probing protocol employing relatively few colors. Cells are cultured in the presence of a mitotic spindle poison, enriching the fraction of cells in metaphase. Locus-specific chromosomal probes, each labeled with a different fluorophore color, are then hybridized to specific locations on the various chromosomes. After hybridization, each chromosome exhibits a unique spectral banding pattern, analogous to a colored bar code. The method takes advantage of spatial separation of the probe colors along a chromosome, using a unique spatial sequence of probe colors on each chromosome to encode chromosomal identity.

U.S. Pat. No.5,539,517 discloses a spectral imaging microscope that, when used in conjunction with the method of U.S. Pat. No. 6,066,459, can produce a multi-spectral composite image of the chromosomes. The composite image is developed by sequentially imaging a field of view (FOV) using an interferometric technique in which each successive image detects a different set of wavelengths. In this manner, images taken at different points in time are used to construct a single composite image of the FOV covering a wide band of wavelengths, enabling the identification of each chromosome based on its characteristic color order. While this method is effective for highly-structured samples that produce repeatable probe sequences such as metaphase chromosomes, it cannot be generally applied to the high throughput multiplex probing of cells since it requires numerous time-consuming sequential images of each FOV. Furthermore, the method requires the size of the probed biological matter to be large relative to the optical resolution of the imaging system in order to resolve the spectral banding pattern. These requirements are incompatible with many applications of biological analysis in which the probed matter is small relative to the spatial resolution of optical microscopy.

These and other limitations of the prior art hinder the study of multiplexed cell-based assays in the drug discovery process, where cells are simultaneously exposed to multiple compounds and the behavior of multiple cellular components are analyzed. Likewise, limitations in the prior art prevent the use of high-throughput diagnostics, such as interphase chromosomal analysis, where more than three or four cellular components must be probed and analyzed simultaneously.

Another example of the state of the art of using singly labeled probes is described in commonly owned U.S. Pat. No. 6,249,341, entitled “Imaging and Analyzing Parameters of Small Moving Objects Such as Cells,” filed on Jan. 24, 2000, the drawings and disclosure of which are hereby specifically incorporated herein by reference. This patent describes generating an image of FISH probes, each of which includes a single binding element and a single signaling element. Each signaling element has an optical signature, such that each different labeled probe is uniquely discriminable by the optical signature of its signaling element. FIG. 2A is illustrative of such an embodiment that images singly-colored probes. A single labeled probe bound to each of three features. Each single labeled probe includes a binding element and a single element. A first probe, made up of signaling element 410a and binding element 412a is associated with a feature 414a. A second probe is made up of signaling element 410b and binding element 412b and is associated with a feature 414a, while a third probe, made up of signaling element 410c and binding element 412c, is associated with a feature 414c. When an image 440 of the object is produced, the spectral signal of each signaling element is spatially separated based on the positions of the features on the object, as indicated by image portion 440a (due to signaling element 410a), image portion 440b (due to signaling element 410b), and image portion 440c (due to signaling element 410c). Each signaling agent is used to identify only a single feature. Thus there is a one to one relationship between the number of signaling elements available and the number of features that can be probed. Notably, the '341 patent does not discuss the use of multiplexed probes within cells or other objects. Again, the total number of probes available to the researcher is typically on the order of the number of unique signaling elements that can be detected with a given apparatus.

Accordingly, it will be apparent that an improved technique is required to overcome the limitations of the conventional approaches discussed above. It is expected that the new approach developed to address these problems in the prior art will also have application to the analysis of other types of cells and biological matter and may be implemented in different configurations to meet the specific requirements of disparate technological fields to which this method can be applied. It would be desirable to provide methods and apparatus that enable more features to be probed using fewer signaling elements, such that the total number of probes available to the researcher is greater than the number of unique signaling elements that can be detected with a given apparatus.

### SUMMARY OF THE INVENTION

The present invention is directed to a method and apparatus for the probing and subsequent simultaneous analysis of a multitude of features in cells, biological matter and other objects. In most embodiments of the analysis system, there is relative movement between an object and the imaging system, although it is contemplated that either (or both) may be stationary. However, in all cases the multispectral imagery necessary to discriminate probed features are acquired simultaneously. In addition, it should also be understood that while much of the following summary and the text that follows recite “an object,” it is clearly contemplated that the present invention is preferably intended to be used with a plurality of objects and is particularly useful in connection with imaging a stream of objects such as cells.

A key feature of the present invention is multiplexing multiple discriminable signaling elements per feature (or object) as a means of increasing the number of different probes that can be employed and discriminated in a cell. This enables the use of more probes within a cell than could otherwise be done with conventional means, thereby enabling the collection of more information from the cell. Each labeled probe includes a probe element that selectively binds to a specific feature (or object), and at least one signaling element. The multiple discriminable signaling elements may be associated with a single binding element specific for the feature, or they may be associated with a set of binding elements, all of which are specific for the same or different components of the feature.

One multiplexing method of the present invention is a binary color encoding scheme in which each feature is distinguished based on its unique color combination of signaling elements. Using C colors, the number of detectable color combinations and therefore the number of uniquely identifiable features P in the binary encoding scheme is:

P−2^{C}−1 (1)

Therefore, the use of four colors yields 15 unique color combinations for discriminating cellular elements. Similarly, using six colors will result in 63 discriminable combinations.

FIG. 1 illustrates the possible color combinations of the binary encoding scheme in four colors, but the present invention is not limited to binary encoding. Also shown is this Figure is an alternative encoding scheme using unique color pairs, where the number of uniquely identifiable features P is given by:

$$P = \frac{{C}^{2} + C}{2} \qquad ( 2 )$$

The present invention can also employ trinary or higher order encoding. Binary encoding effectively employs two intensities for each color; a color is either present or absent from a feature. More than two intensities can be employed to increase the number of possible color combinations. In this case the total number of detectable features P using C colors with I intensities is:

p=I^{C}−1 (3)

As shown in Equation (3), four colors and four intensities results in 255 uniquely distinguishable combinations. In the present invention, quantitative intensity measurement is useful even in a binary encoding scheme by enabling the discrimination of overlapping features that share a common color or colors within a cell. For example, if two red-containing features appear at the same location, the intensity of red will be increased relative to the other colors employed in one or the other of the features.

A first embodiment of the present invention is directed to a set of labeled probes, each labeled probe including one binding element that selectively binds to a feature and multiple signaling elements. Each probe has an optical signature, such that each different labeled probe is uniquely discriminable by the composite optical signature produced by its signaling elements. The plurality of signaling elements provide a multiplexed signal, enabling more features to be probed using a smaller set of signaling elements. Note that as illustrated in FIG. 2A, a one-to-one relationship exists between the number of features that can be probed, and the number of unique signaling elements available. In the present invention, the number of features that can be probed exceeds the number of unique signaling elements available. FIG. 2B illustrates this encoding embodiment with binary coded probes, while FIG. 2C illustrates this embodiment with intensity coded probes. Note that the multiple signaling elements on each probe can be identical to each other, or can be a combination of different signaling elements.

In a second embodiment of the present invention, an optical signal is generated by a plurality of labeled probes bound to the feature, each labeled probe including the same binding element and at least one signaling element, each signaling element having an optical signature, such that each different feature is uniquely discriminable by the composite optical signature of its plurality of bound probes. Note that while singly labeled probes similar to those illustrated in FIG. 2A are employed in this embodiment, the present method of using such probes is distinguishable. In this embodiment each different feature or object is capable of binding multiple singly-colored probes, each having an identical binding element, in sufficiently close physical proximity that the imaging system is unable to spatially resolve the different probes. As a result, the image of the feature or object contains a multiplexed signal very similar to that which would be produced by a single multi-colored probe of the first embodiment described above.

For example, a particular chromosome may contain repetitive tandem DNA sequences unique to that chromosome, each repetitive DNA sequence binding a single probe. Since the tandem repeats are immediately adjacent and cannot be resolved spatially, and each repeated sequence can randomly bind one of a plurality of singly-colored probes in the mixture, the imaging system detects multiple colors emitted from the chromosome at each pixel location in the image. FIG. 2D illustrates this embodiment with binary coded probes. Intensity coding can be implemented in this embodiment by varying the proportion of the differently-colored probes. For example, if a mixture of signaling elements A, B and C are distributed equally (i.e. 33.3% A, 33.3% B, and 33.3% C) among a plurality of singly-colored probes binding to feature X, and signaling elements A, B and C are distributed unequally (50% A, 25% B, 25% C) among a plurality of labeled probes binding to feature Y, the multiplexed signal from feature X can be discriminated from the multiplexed signal from feature Y on the basis of the different relative intensities of the colors, even though the same colored signaling elements are employed in both cases. This concept is illustrated in FIG. 2E.

In a third embodiment of the present invention, an optical signal is generated by a plurality of labeled probes bound to the feature. The labeled probes employed in this embodiment are once again singly labeled probes, each labeled probe including a different binding element and at least one signaling element, each signaling element having an optical signature. Once again, such probes are employed in an unconventional fashion to generate a multiplexed signal. In the present embodiment, individual probes binding to a specific feature will include a plurality of different binding elements, rather than all probes having the same binding element. Each different feature becomes uniquely discriminable by the composite optical signature of its plurality of bound probes. For example, each of the different binding element may target a different domain or subunit of a given protein. Because individual protein molecules are below the resolution limit of the optical detection system, each protein will appear to emit a single multiplexed signal despite the fact that the signal arises from multiple signaling elements, each of which may be physically attached to different binding elements at different locations on the protein. Binary coded and intensity coded probes of this embodiment are illustrated in FIGS. 2F and 2G, respectively.

Preferably, each different signaling element is uniquely identifiable based on a wavelength of light associated with that signaling element. In some embodiments, each different signaling element is uniquely identifiable based on an intensity of a wavelength of light associated with that signaling element.

In all embodiments of the present invention, multiplexed probes within a cell are discriminated by simultaneously acquiring a set of images of the object, each image being of a different spectral band and having sufficient spatial resolution to resolve the locations of features within the object. Corresponding locations are analyzed across the image set to determine the spectral composition, and therefore the probe identity, at each location in the object. FIG. 3 illustrates the set of images projected onto the detector in one embodiment of the invention. This embodiment contains six discrete color zones on the detector, from violet on the left side, to red on the right side. In this example, the violet portion of the spectrum is used to image scattered laser light while the five color zones to the right are used for probe detection.

In the present invention, the set of single-color images are acquired simultaneously, either on a single detector or on multiple detectors. Simultaneity is important for several reasons. First, the present invention is intended to be applicable to the imaging of living cells and other dynamic objects that can change internal structure over time. Without simultaneity across all single-color data, the position of probes may change between images, precluding correlation of probe position from one single-color image to the next. Secondly, probe spectra and intensities can change over time due to biological activity or exposure to light. As in the case of probes losing register between images, probes changing color or intensity can hinder discrimination. Finally, simultaneous probe imaging enables high throughput analysis.

Another important aspect of the present invention is directed to a system for labeling and analyzing a plurality of different features associated with an object. Such a system includes a plurality of labeled probes, each different labeled probe having a uniquely identifiable image. Each labeled probe includes a binding element selected from among a plurality of different binding elements included in the system, each different binding element selectively binding to a different feature, and at least one signaling element selected from among a plurality of different signaling elements included in the system. Each signaling element has a unique optical signature, such that at least two labeled probes that bind to different features include at least one common signaling element. Finally, the system includes means for imaging the object, the means identifying each different type of labeled probe bound to one of the plurality of different features associated with the object.

In one embodiment, the means includes a collection lens disposed so that light traveling from the object passes through the collection lens and is focused along a collection path, and a dispersing element that receives the light from the collection lens and disperses the light into a plurality of light beams, as a function of a plurality of different discriminable characteristics of the light, the plurality of different discriminable characteristics being indicative of the plurality of different signaling elements. Such means also includes at least one pixilated detector, and an imaging lens that focuses each of the plurality of light beams on the at least one pixilated detector, producing a respective image corresponding to each of the plurality of light beams, the at least one pixilated detector providing an output signal for each respective image, each output signal indicating whether a different one of the plurality of signaling elements is associated with the object. Finally, such a system includes a signal processor coupled to receive the output signals from the at least one pixilated detector, the signal processor processing the output signals to determine which labeled probes are bound to features associated with the object.

Preferably, the dispersing element includes one of a dichroic filters and a prism, and the at least one pixilated detector includes a time delay integration (TDI) detector.

In another embodiment, the means includes at least one light source for illuminating the object, and a collection lens disposed so that light traveling from the object passes through the collection lens and travels along a collection path. Such means also employs a dispersing element that receives the light from the collection lens and disperses the light into a plurality of light beams, as a function of a plurality of different discriminable characteristics of the light, the plurality of different discriminable characteristics being indicative of the plurality of different signaling elements, and at least one pixilated detector. An imaging lens is employed to focus each of the plurality of light beams on the at least one pixilated detector, producing a respective image corresponding to each of the plurality of light beams, the at least one pixilated detector providing an output signal for each respective image, each output signal indicating whether a different one of the plurality of signaling elements is associated with the object, and a signal processor is coupled to receive the output signals from the at least one pixilated detector, the signal processor processing the output signals to determine which labeled probes are bound to features associated with the object.

In still another embodiment, the means includes a collection lens disposed so that light traveling from the object passes through the collection lens and travels along a collection path, and a plurality of light reflecting elements disposed in the collection path, each light reflecting element reflecting light of a different predefined characteristic, and passing light that does not have that predefined characteristic, the signaling elements in each object determining the characteristics of light traveling along the collection path, each light reflecting element being positioned at a different location with respect to the collection path to reflect light of a specific predefined characteristic in a direction different from that of other light reflecting elements, each light reflecting element being positioned along an axis of the collection path, such that passing light not reflected by a preceding light reflecting element reaches a last light reflecting element. Such means also includes at least one pixilated detector disposed to receive light that has been reflected by each of the light reflecting elements, the at least one pixilated detector comprising a plurality of pixilated regions, each pixilated region producing an output signal that is indicative of at least one characteristic of the signaling elements and thus indicative of labeled probes. Finally, a signal processor is coupled to receive the output signals from the plurality of regions, the signal processor processing the output signals to determine which labeled probes are associated with the object.

Other aspects of the invention are directed toward methods whose steps are generally consistent with the elements of the apparatus described above.

### BRIEF DESCRIPTION OF THE DRAWING FIGURES

The foregoing aspects and many of the attendant advantages of this invention will become more readily appreciated as the same becomes better understood by reference to the following detailed description, when taken in conjunction with the accompanying drawings, wherein:

FIG. 1 is a graphic illustration showing a plurality of multiplexed coding schemes that can be employed in the present invention to increase the number of components that are simultaneously probed;

FIGS. 2A (Prior Art) is a graphic illustration showing how probes, each including only a single color and a single binding element, bind to different features of an object, producing a related set of images on a detector;

FIGS. 2B-2G are a series of graphic illustrations showing different configurations of probes, each in accord with the present invention, bound to the same features of FIG. 2A, thereby producing a set of images on a detector;

FIG. 3 is a graphic illustration showing a set of images projected upon a detector in an embodiment of the present invention using six colors, including four colors for the probes;

FIG. 4 is an isometric view of an embodiment of an imaging system in which multiple legs are employed for prism-based spectral decomposition and imaging to collect light signals from multiple perspective positions;

FIG. 4A is a greatly enlarged section of a cuvette, a portion of which is broken away to show cells conveyed in a flow past the imaging system of FIG. 4;

FIG. 5 is an isometric view of an embodiment of an imaging system for collecting multiplexed probe imagery from objects affixed to a solid substrate, in which spectral decomposition is accomplished with a filter stack, and in which a slit is used for spatial filtering of extraneous light;

FIG. 6 is an isometric view illustrating a plurality of different illumination modes of a prism-based embodiment of an imaging system for collecting multiplexed probe imagery from objects in flow;

FIG. 7 is an alternative embodiment to that of FIG. 6, in which a second set of imaging components and detector are included for monitoring light from an object, to collect three dimensional imagery;

FIG. 8 is a schematic diagram illustrating the optical convolution of a narrow FISH emission spectrum by the present invention, to image a non-multiplexed FISH probe in a cell;

FIG. 9A is a schematic diagram illustrating the optical convolution of two narrow FISH emission spectra by the present invention, to image a multiplexed FISH probe in a cell;

FIG. 9B is a schematic diagram like that in FIG. 9A, but imaging a multiplexed FISH probe in which the two narrow emission spectra have substantially different intensities;

FIG. 10 is a schematic diagram illustrating how for a wider FISH emission spectrum, a digital deconvolution is provided by the present invention to resolve the image of a non-multiplexed FISH probe;

FIG. 11 is a schematic diagram illustrating how for a wider FISH emission spectrum, a digital deconvolution is provided by the present invention to resolve the images of a multiplexed FISH probe having two FISH emission spectra;

FIG. 12 is a functional schematic block diagram of the electronics system used to process the signal produced by a time delay and integration (TDI) detector in the present invention;

FIG. 13 is a schematic diagram illustrating how an imaging system in accord with the present invention is used with both multiplexed and non-multiplexed probes to determine whether a cell is from a male or female;

FIG. 14 is a plan view of an alternate embodiment that employs a spectral dispersion component comprising a plurality of stacked dichroic filters that spectrally separate the light incident on different portions of a detector;

FIG. 15 is an X-Y plot of several typical passbands for the dichroic filters employed in the embodiment shown in FIG. 14;

FIG. 16 is a schematic illustration of a detection filter assembly that may optionally be placed in front of the detector in the embodiment of FIG. 18 to further suppress out-of-band light;

FIGS. 17A-17E are X-Y plots of transmission vs. wavelength corresponding to different passbands of the filter segments of the detection filter assembly of FIG. 16;

FIG. 18 is a plan view of another embodiment of the configuration of FIG. 14, wherein the spectral dispersion filter system comprises a plurality of dichroic cube filters orientated at various angles to create the spectral dispersing effect;

FIG. 19 is a schematic isometric view of another embodiment of an imaging system for implementing the present invention, in which the spectral emission is not convolved with the image and in which the spectral decomposition occurs in an axis perpendicular to flow through the use of separate dichroic filters, imaging lenses, and detectors for each spectral region;

FIG. 20 is a schematic isometric view of yet another embodiment similar to that of FIG. 19, but in which spectral decomposition occurs in an axis that is generally parallel to a direction of motion of a substrate carrying an object; and

FIG. 21 is a schematic plan view of a spectrally segmented detector for use in detecting and imaging light of several different spectral compositions.

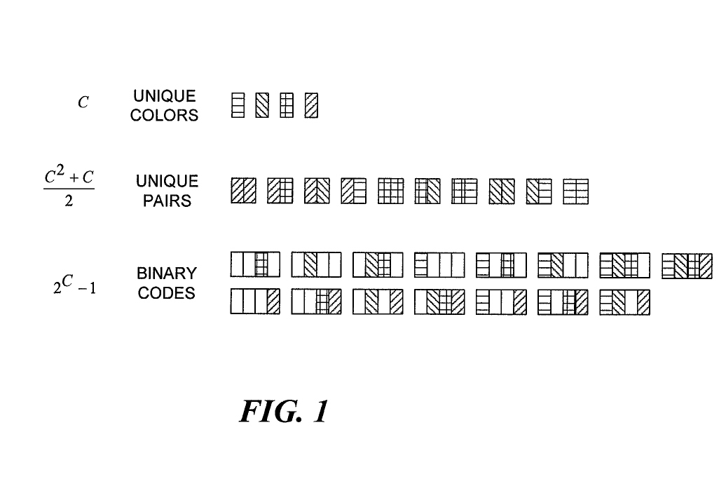

### DESCRIPTION OF THE PREFERRED EMBODIMENT

It is anticipated that the present invention may be particularly applicable to the analysis of multiplexed probe signals from within cells, but it should be emphasized that this invention is applicable to the optical analysis of objects in general. Signaling elements binding to a feature of a cell or other object are multiplexed in the present invention to increase the number of combinations of uniquely discriminable probe entities. This enables a larger number of different features to be probed using fewer signaling elements than there are features. Although several embodiments exist to achieve the multiplexing function, in each embodiment, a given signaling element is usable for more than one purpose. For example, if a fluorophore is used as the signaling element, the same fluorophore can be used to label several different probes and therefore may appear in multiple locations within the cell or other object. The identity of a component within the object will be determined by the combination of fluorophores present at a given location. Likewise, several embodiments of apparatus capable of reading the plurality of uniquely discriminable probes on an object are disclosed for the present invention. However, in all such embodiments, the apparatus simultaneously acquires imagery of all signaling events.

### New Method for Probe Multiplexing

The present invention analyzes the spectral content in every pixel of an image by spectrally decomposing the image into multiple single-color images, one image per color. Any cellular component that is labeled with a unique color combination can therefore be discriminated. There are a number of methods of labeling a cellular component with multiple colors.

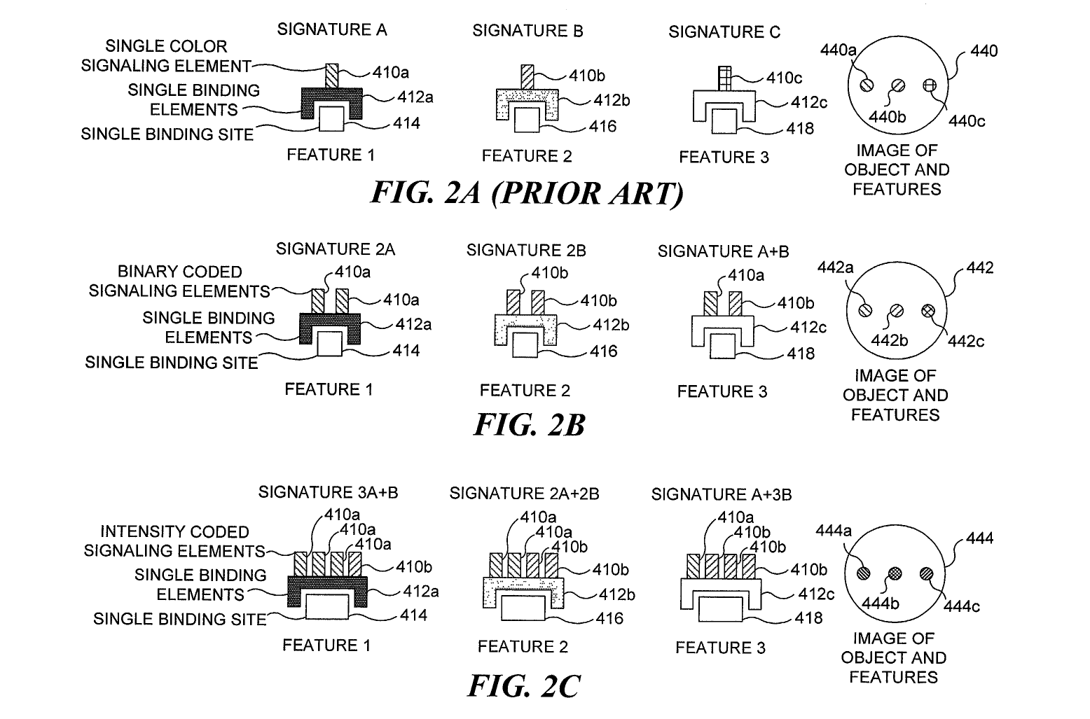

A first probe labeling method, schematically illustrated in FIGS. 2B and 2C, is to bind multiple fluorescent dye molecules (signaling elements) to a single binding element, directly embodying the probe's multiplex color code on the probe itself. Various ratios of the different color fluorescent dyes can be bound to the binding element if intensity is included in the encoding scheme. When the binding element of the labeled probe binds to its cellular target and the fluorescent molecules are excited, multiple fluorescent colors are emitted from each probe. As a result, this method can be employed to probe features within a cell that fall below the resolution limit of the imaging system, as long as the spectra from each pixel can be determined. In this method, an image pixel's spectral signature is independent of the number of labeled probes represented in that pixel.

In referring to FIGS. 2A-2G, it should be noted that each Figure shows a plurality of features associated with an object, and one or more labeled probes bound to at least a portion of each feature. An image of the object is also shown for each Figure. For each Figure, for purposes of simplicity in these examples, the object has arbitrarily been defined as including three features of interest. The image in each Figure includes optically discriminable signatures provided by signaling elements included in labeled probes bound to each feature, but the individual signaling elements themselves are not spatially discriminable in the images. The present invention optically images an object whose features have been marked. Each feature (and a multiplex or composite of the optical signatures of all the signaling elements bound to that feature) is evident in the image. While it is possible that some features are disposed so closely together for two features to “blur” together, often features of interest can be spatially distinguished in an image of the object.

In the prior art, each individual signaling element was required to have a one-to-one relationship to either a specific feature, or a specific binding site on that feature. To be able to distinguish one feature from another, each signaling element had to be spatially resolved (i.e., spatially distinguished at distinct locations on the feature) from any other signaling element bound to the same feature. If two or more signaling elements bound to a feature could not be spatially resolved, the prior art did not enable the feature to be distinguished from other features that were marked using the same signaling elements for identification.

In the present invention, it is not necessary to spatially resolve the signaling elements associated with the same feature, as long as each feature can be spatially resolved from other features on the object. In the prior art, the location and identity of each signaling element associated with a feature is used to identify the feature. In the present invention, the specific spatial location of each signaling element associated with a feature is irrelevant. Consider an image 442 of an object as illustrated in FIG. 2B. The object includes three features, as indicated by features 414, 416, and 418. In image 442, each feature can be spatially distinguished from the other features, as is indicated by image portions 442a, 442b, and 442c. Thus, image portion 442a corresponds to feature 414, image portion 442b corresponds to feature 416, and image portion 442c corresponds to feature 418. As is clearly shown in FIG. 2B, two signaling elements 410a are associated with feature 414. Referring to image 442, specifically image portion 442a, those two signaling elements are not spatially distinguishable. Both signal elements contribute to provide a multiplexed optical signature that is optically distinguishable, even though the separate signaling elements are not individually spatially discriminable. Note that the optical signature for feature 414 of FIG. 2B is identified with the reference “2A,” each signaling element 410a contributing an optical signature identified by the reference “1A.” In FIG. 2A, the optical signature of feature 414 of is identified as “1A,” because in FIG. 2A only a single signaling element 410a is associated with feature 414. When comparing image portions 440a and 442a (in FIGS. 2A and 2B), the optical signatures in each image can be distinguished based on the intensity of each optical signature (“1A” versus “2A”). However, the ability to optically distinguish optical signature 2A from optical signal IA is not a function of the ability to spatially discriminate the signaling elements responsible for the optical signatures. All that is required is that the composite optical signature from one feature be distinguishable from a composite optical signature of a different feature, where each optical signature is a function of the optical signaling elements associated with each feature. Details of the method and apparatus employed to produce such images are provided below.

Referring once again to FIG. 2B, a first probe associated with feature 414 has two signaling elements 410a coupled to binding element 412a. A second probe having two signaling elements 410b coupled to binding element 412b is thus bound to feature 414. Note that feature 418 is uniquely identified without requiring the use of another signaling element. Feature 418 is identified by a third probe that includes one signaling element 410a and one signaling element 410b, each of which are coupled to binding element 412c. The composite optical signal identifying feature 418 is “1A+1B.” The order of the signaling elements relative to feature 418 is irrelevant. As shown, signaling element 410a is disposed to the left of signaling element 410b. If their positions were reversed, the composite optical signature would be “1B+1A.” Referring now to image portion 442c, note that because image portion 442c does not spatially distinguish the relative positions of signaling elements 410a and 410b, it cannot be determined if the spatial order of composite optical signature is “1A+1B” or “1B+1A.” But in the present invention, feature 418 is positively identified by either “1A+1B” or “1B+1A,” whereas in prior art, a feature could not be positively identified unless the spatial position of “A” relative to “B” could be determined. The use of spatially independent optical signatures enables imaging systems to be employed that need not spatially resolve each signaling element. One benefit of the imaging system used in the present invention is that it can operate very rapidly, enabling features to be identified much more quickly. By providing a multiplexed signal that does not need to be spatially resolved, it is contemplated that many new applications will be identified for this invention.

In FIG. 2C, the multiplexed signal employs intensity to distinguish between probes. The probe coupled to feature 414 has three signaling elements 410a and one signaling element 410b coupled to binding element 412a. The probe associated with feature 416 has two signaling elements 410a and two signaling elements 410b coupled to binding element 412b. Finally, feature 418 is identified with a probe having one signaling elements 410a and three signaling elements 410b coupled to binding element 412c. Referring to an image 444 of the object, note that both signaling elements 410a and 410b are each represented in the image portions corresponding to the different features. However, the different features are still distinguishable based on the unique optical signatures different combinations of the same signaling elements produced for each feature. An image portion 444a is produced by the three signaling elements 410a and the one signaling element 410b associated with feature 414. An image portion 444b is produced by the two signaling elements 410a and two signaling elements 410b associated with feature 416. Finally, an image portion 444c is produced by the one signaling element 410a and the three signaling elements 410b associated with feature 418. Note that the each image portion can be positively distinguished based on the intensities of the common signaling elements. Clearly, using intensity of a waveband in the multiplexed signal to distinguish between probes enables even more different features to be identified using a limited pool of signaling agents.

A second probe labeling method is used to produce a mixture of probes that each include identical binding elements, each binding element being associated with a single fluorescent molecule. The different fluorescent molecules employed in the mixture of singly-labeled probes determine the feature's multiplex color code. A plurality of such probes bind to each feature of a cell or object, as illustrated in FIG. 2D, which shows binary encoding (three features being identified using only two different signaling elements).

In FIG. 2D, features 424, 426 and 428 each include three binding sites, respectively labeled 424a-424c, 426a-426c, and 428a-428c. Thus, three probes are bound to each feature. The three probes bound to each feature of FIG. 2D have the same binding element, and include a single signaling element. Because a plurality of individual probes are bound to each feature, a multiplexed signal is provided for each feature, even though each labeled probe includes only a signal signaling element. The three labeled probes bound to feature 424 each have one signaling element 410a coupled to binding element 412a, while the three labeled probes bound to feature 426 each have one signaling element 410b coupled to binding element 412a. Unlike the labeled probes associated with features 424 and 426, the labeled probes associated with feature 428 are not identical, even though each of these three labeled probes includes binding element 410c. The labeled probes bound to binding sites 428a and 428b of feature 428 each include signaling element 410a, while the labeled probe bound to binding site 428c of feature 428 includes signaling element 410b.

In an image 446 of the object, again three distinct optical signatures are produced using only two signaling elements (signaling elements 410a and 410b). An image portion 446a is produced by the three signaling elements 410a that are coupled to feature 424 with three different labeled probes. It should be understood that image portion 446a would be the same regardless of whether the three signaling elements 410a associated with feature 424 were coupled to the feature with a single labeled probe (i.e. one labeled probe consisting of three signaling elements 410a and one binding element 412a) or the three different labeled probes shown in FIG. 2D. An image portion 446b is produced by the three signaling elements 410b associated with feature 426. Finally, an image portion 446c is produced by the two signaling elements 410a and the one signaling element 410b associated with feature 428. Note that each image portion 446a, 446b, and 446c are very closely related to image portions 442a, 442b, and 442c of FIG. 2B. It should be understood that even if image portion 446a (due to three signaling elements 410a) and image portion 442a (due to two signaling elements 410a) were present in the same image, the different intensities of the signals would enable the different image portions (and hence the different features) to be distinguished, as long as the image portions do not overlap. Because features represent physical structures of an object, the image portions representing the signaling elements bound to the different features will generally be spatially separated on the resulting image.

Various ratios of the different singly-labeled probes can be mixed to use intensity in the encoding scheme, as shown in FIG. 2E. In FIG. 2E, features 420, 422, and 423 each include four binding sites, respectively labeled 420a-420d, 422a-422d, and 423a-423d. Thus, four probes are associated with each feature. The four probes associated with each feature of FIG. 2E use the same binding element, and each includes only a single signaling element. As before, even though each probe includes only a single signaling element, because a plurality of individual probes are bound to each feature, a multiplexed signal is produce to identify each feature. The four labeled probes bound to feature 420 each have one signaling element coupled to binding element 412a; the four labeled probes bound to feature 422 each have one signaling element coupled to binding element 412b; and the four labeled probes bound to feature 423 each have one signaling element coupled to binding element 412c. In this embodiment, none of the features are identified using only a single type of signaling element, unlike the previous embodiments of FIGS. 2A-2D. The labeled probes bound to bindings site 420a, 420b, and 420c of feature 420 each include signaling element 410a, while the labeled probe bound to binding site 420d of feature 420 includes signaling element 410b. The labeled probes bound to bindings site 422a and 422b of feature 422 each include signaling element 410a, while the labeled probe bound to binding sites 422c and 422d of feature 422 each include signaling element 410b. The labeled probes bound to binding site 423a of feature 423 includes signaling element 410a, while the labeled probes bound to binding sites 423b, 423c, and 423d of feature 423 each include signaling element 410b. Even though the optical signatures of the labeled probes bound to each feature all include signaling elements 410a and 410b, the intensities of the signaling elements in the optical signals associated with each feature are different, enabling each feature to be readily distinguished from the others.

Referring to an image 448 of the object in FIG. 2E, an image portion 448a is produced by the three signaling elements 410a and one signaling element 410b that are coupled to feature 420 with four different labeled probes. Again, image portion 448a would be the same regardless of whether the three signaling elements 410a and one signaling element 410b associated with feature 420 were coupled to the feature with a single labeled probe (i.e. one labeled probe consisting of three signaling elements 410a, one signaling element 410b, and one binding element 412a) or the four different labeled probes shown in FIG. 2E. This is clearly shown by comparing FIG. 2C with FIG. 2E. An image portion 448b is produced by the two signaling elements 410a and the two signaling elements 410b associated with feature 422. Finally, an image portion 448c is produced by the one signaling element 410a and the three signaling elements 410b associated with feature 423.

In such an embodiment, the resulting optical signature in a pixel included in an image of the object is theoretically dependent on the number of labeled probes represented at that pixel. If only a subset of the colors in the probe mixture bind to a region of the cell, the probe's multiplex color code will not be accurately represented in the optical signature at that pixel. However, it is generally true that even very small structures within the cell will bind to numerous labeled probes, and each pixel in the image of the object or cell will therefore integrate light from each of the labeled probes, making it very likely that each color in a probe mixture will be represented in the optical signature at a pixel of the image. An extension of this method does not require that the different binding elements of the labeled probes be identical, as long as they each bind to a cellular structure that is below the resolution limit of the optical analysis system. Under these conditions, the analysis system can still determine the color code of the probe on a pixel by pixel basis.

A third labeling method relies not on determining the optical signature of a particular pixel, but instead on determining the optical signature of a cellular structure that spans several pixels. In this third method, several different probes specific to different locations on the same cellular structure are employed. As in the second method, each cellular structure is labeled with a mixture of singly-labeled probes. Upon imaging, the cellular structure is segmented by its overall morphology and identified by the multiplex color code of its constituent probe labels. As is illustrated in FIGS. 2F and 2G, different binding elements are used to identify the same feature.

In FIG. 2F, features 425, 427, and 429 each include three binding sites, respectively labeled 425a- 425c, 427a- 427c, and 429a- 429c. Thus, three probes are associated with each feature. Each of the three probes associated with each feature of FIG. 2F have different binding elements, and include one of two different signaling elements. Specifically, the labeled probe bound to binding site 425a of feature 425 includes a signaling element 410a coupled to binding element 412a; the labeled probe bound to binding site 425b of feature 425 has signaling element 410a coupled to binding element 412b; and the labeled probe bound to binding site 425c of feature 425 has signaling element 410a coupled to binding element 412c. Similarly, the labeled probe bound to binding site 427a of feature 427 includes signaling element 410b coupled to binding element 412d; the labeled probe bound to binding site 427b has signaling element 410b coupled to binding element 412e; and the labeled probe bound to binding site 427c includes signaling element 410b coupled to binding element 412f. Referring to feature 429, the labeled probe bound to binding site 429a has signaling element 410a coupled to binding elements 412a; the labeled probe bound to binding site 429b has signaling element 410a coupled to binding element 412b; and the labeled probe bound to binding site 429 c includes signaling element 410b coupled to binding element 412d. Using only a limited set of signaling elements, features including a plurality of different binding sites can thereby be readily identified.

In a related image 450 of the object, the three distinct optical signatures are still produced using only two signaling elements (signaling elements 410a and 410b). An image portion 450a is produced by the three signaling elements 410a that are coupled to feature 425 with three different labeled probes, each of which include a different binding element as noted above. An image portion 450b is produced by the three signaling elements 410b associated with feature 427. Finally, an image portion 450c is produced by the two signaling elements 410a and the one signaling element 410b associated with feature 429. Note that the image portions 450a 450c of FIG. 2F are identical to (that is they cannot be distinguished from) the image portions 446a-446c from FIG. 2D. the set of labeled probes in FIG. 2D are different from the set of labeled probes in FIG. 2F only in their respective binding elements. The signaling elements are identical, as indicated by the respective image portions.

In FIG. 2G, features 430, 432, and 434 each include four binding sites, respectively labeled 430a-430d, 432a-432d, and 434a-434d, and four probes associated with each feature. None of the four probes associated with each feature of FIG. 2F includes the same binding element, and no feature is identified using only a single signaling element in all of the probes bound to that feature. Despite the plurality of different binding elements required in this embodiment, only two different signaling elements are required to uniquely identify all three features. While more features would require more signaling elements, clearly the required one-to-one correspondence between each feature and each different signaling element of the prior art (FIG. 2A) is avoided.

The four labeled probes bound to feature 430 are configured as follows. The labeled probe bound to binding site 430a has signaling element 410a coupled to binding element 412a; the labeled probe bound to binding site 430b includes signaling element 410a coupled to binding element 412b; the labeled probe bound to binding site 430c has signaling element 410a coupled to binding element 412c; and the labeled probe bound to binding site 430d includes signaling element 410b coupled to binding element 412d. The four labeled probes bound to feature 432 are configured as follows. The labeled probe bound to binding site 432a includes signaling element 410a coupled to binding element 412a; the labeled probe bound to binding site 432b has signaling element 410a coupled to binding element 412b; the labeled probe bound to binding site 432c includes signaling element 410b coupled to binding element 412d; and the labeled probe bound to binding site 432d has signaling element 410b coupled to binding element 412e. Finally, in regard to feature 434, the labeled probe bound to binding site 434a includes signaling element 410a coupled to binding element 412a; the labeled probe bound to binding site 434b has signaling element 410b coupled to binding element 412d; the labeled probe bound to binding site 434c includes signaling element 410b coupled to binding element 412e; and the labeled probe bound to binding site 434d includes signaling element 410b coupled to binding element 412f.

Referring to an image 452 of the object shown in FIG. 2F, an image portion 452a is produced by the three signaling elements 410a and one signaling element 410b that are coupled to feature 430 with four different labeled probes, each of which includes a different binding element. An image portion 452b is produced by the two signaling elements 410a and the two signaling elements 410b that are coupled to feature 432 with four different labeled probes, each of which includes a different binding element. Finally, an image portion 452c is produced by the one signaling element 410a and the three signaling elements 410b associated with feature 434. Note that the image portions 452a-452c of FIG. 2G are identical (that is they cannot be distinguished from) to the image portions 448a-448c from FIG. 2E. The set of labeled probes in FIG. 2G are different from the set of labeled probes in FIG. 2E only in their respective binding elements. The signaling elements are identical, as indicated by the respective image portions.

A common feature in all of the embodiments discussed in reference to FIGS. 2B-2G is that at least one labeled probe binding to a feature of an object includes a signaling element that is also included in a labeled probe binding to a different feature of the object. Also, the ability of the imaging systems disclosed herein to discriminate spectrally and by intensity, the simultaneous multiplexed contributions of a plurality of probes, enables the probes to be readily detected, so that the features to which they are bound can be efficiently identified.

### New Method for Analyzing Probe Multiplexes

A new flow imaging system and method for analyzing probe multiplexes overcomes the problems experienced in the prior art for carrying out this task and adds new capabilities to the analysis and handling of cells that are provided with probes. The flow imaging system enables the discrimination of the different multiplexed probes within a cell and therefore enables the analysis of many biological parameters at a time. By handling cells in suspension and using hydrodynamic focussing, billion-count cell samples can easily be moved through the FOV of the flow imaging system at high rates. Existing non-imaging flow cytometers cannot be used with this multiplex scheme because within a given cell, different probes having any colors in common cannot be distinguished from each other without spatial information. Existing imaging systems cannot be used with this multiplex scheme, because the scheme requires multiple images, each of a different color, to be acquired simultaneously.

The present invention for analyzing cells in flow includes subsystems that carryout the tasks of: optical signal collection and spectral decomposition, pixilated detection, illumination, cell velocity detection, and sample handling. It will be apparent to those of ordinary skill in the art that, depending upon the probing and image acquisition methods applied in the present invention, one or more of these systems may not be required.

An important aspect of the present invention lies in the ability to simultaneously discriminate the location of the various fluorescent emission spectra produced by probes in or on a cell. This ability enables the rapid determination of the type and location of each probe after a cell has passed through the FOV. Hydrodynamic focusing ensures that the cells are at or near the focal plane of the imaging system and are lined up in a single file along the axis of flow. The imaging system can be constructed to view the cells from multiple angles to produce image data of a greater fraction of the cell's surface and volume, to enable the construction of a three-dimensional representation of the cell. The present invention takes advantage of the single file orientation of the cells in order to spectrally decompose the signal from each cell in the axis perpendicular to flow and then forms images of the probes within a cell onto a single or multiple detectors. The location of the images on the detector is determined by the spectral content of the signal emitted from a probe as well as the spatial position of the probe with respect to the cell. There are four embodiments of the present invention that accomplish the spectral decomposition and imaging of the probes in this manner. cl First Embodiment for Spectral Decomposition and Imaging

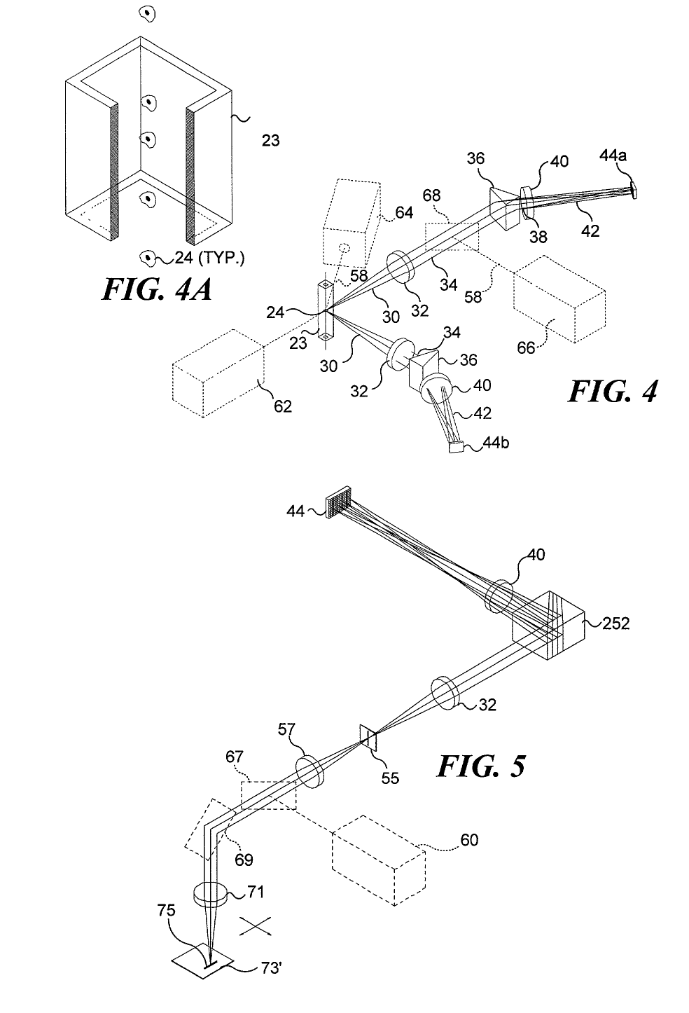

A first embodiment of the present invention is shown in FIG. 6, which is also included in U.S. patent application Serial No. 09/490,478, entitled, “Imaging and Analyzing Parameters of Small Moving Objects Such as Cells,” filed on Jan. 24, 2000, the drawings and disclosure of which are hereby specifically incorporated herein by reference. In this previously filed application, there is no discussion of identifying multiplexed reporters within cells or other objects. However, the following describes how the apparatus disclosed in this previously filed application can be employed for spectral decomposition and imaging of objects that include a plurality of multiplexed reporters. In regard to the present invention, a cell provided with multiplexed probes is simply a specific type of object. Note that where there is any variance between the description in any document incorporated herein by reference and the present disclosure, the present disclosure takes precedence.

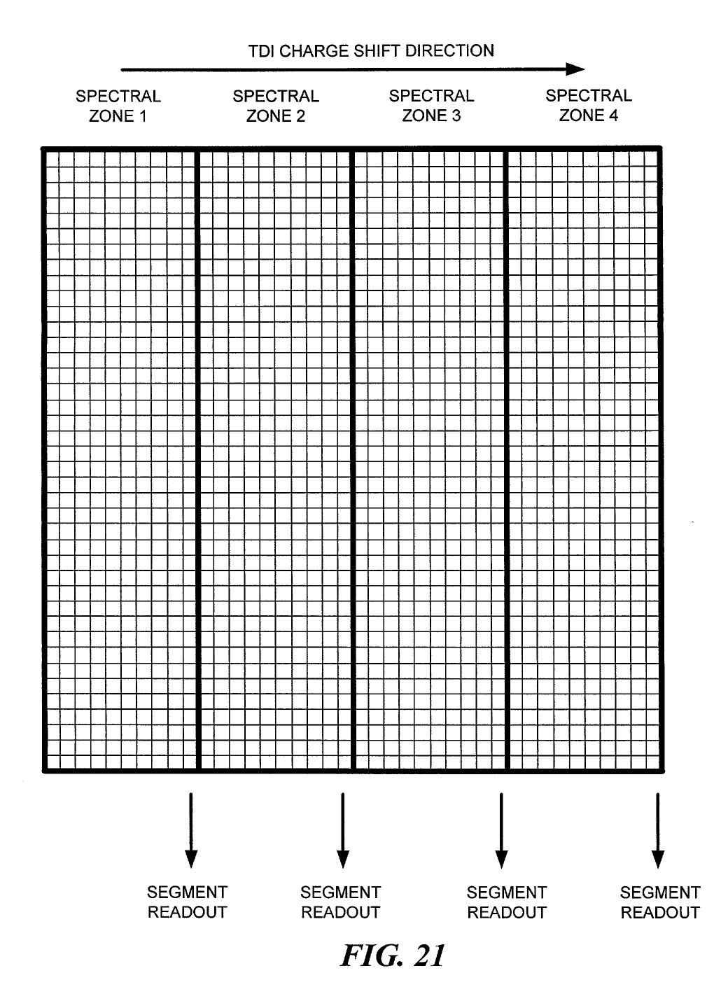

As shown in the FIG. 6, a column of cells 22 is hydrodynamically focused to a well-defined region; light from the cells is collimated by passing through a collection lens 32. There is relative movement between the cells and the imaging system illustrated in the Figure. As illustrated in this example, the cells are moving in the column, past the imaging system. The light from the cells travels along a collection path 30. A spectral dispersing element 36 disposed in the collection path spectrally disperses the collimated light that has passed through the collection lens in a plane that is substantially orthogonal to a direction of relative movement between the cells and the imaging system, producing spectrally dispersed light. An imaging lens 40 is disposed to receive the spectrally dispersed light, producing an image from the spectrally dispersed light. Also included is a pixilated detector 44, disposed to receive the image produced by the imaging lens. Details of one exemplary pixilated detector are schematically illustrated in FIG. 21. As the movement of the cells relative to the imaging system occurs, the image of the object produced by the imaging lens moves from row to row across the pixilated detector. As will be described later, the pixilated detector may be a TDI-type detector or a frame type detector.

As a result of light collimation by the collection lens in this embodiment of the imaging system, all light emitted from a first point in the cell travels in substantially parallel rays. Light emitted from a second point in the cell will also travel in substantially parallel rays, but at a different angle relative to light from the first point. In this manner, spatial information in the cell is transformed by the collection lens into angular information in the collection path. The spectral dispersing element acts on the collimated light in the collection path, such that different spectral components of the collimated light leave the spectral dispersing element at different angles, in a plane substantially orthogonal to the direction of relative movement between each cell and the imaging system. In this manner, both spatial and spectral information in a cell are transformed into angular information. The imaging lens acts on the light from the dispersing element to transform different light angles into different positions on the detector. Spatial information is preserved by the system, since light from the different positions in the cell is projected to different positions on the pixilated detector, in both axes. In addition, light of different spectral composition that originates from the cell is projected to different positions on the detector in an axis substantially orthogonal to the movement. In this manner, the spatial information from the cell is preserved, while simultaneously collecting spectral information covering a large bandwidth at high resolution.

When used for multiplexed probe identification in accord with the present invention, this apparatus provides substantial utility in resolving probe location and spectra on the detector, even when the probes are disposed in spatially close relationship within a cell. When spectral imaging occurs in the present invention, the spatial distribution of light in the cell is convolved with the spectral distribution of that light to produce the image of the cell at the detector. This convolution can result in blurring in the dispersion axis, depending on the spectral bandwidth of the light. Narrow spectral bandwidths will result in little or no blurring depending on the spectral resolution of the system. In the present invention, it is contemplated that the spectral resolution will be approximately 3 nm per pixel, with a spatial resolution in object space of approximately 1 micron. However, the spatial and spectral resolution can be adjusted to match the requirements of the particular application, and the exemplary specifications set forth above should not be considered limiting.

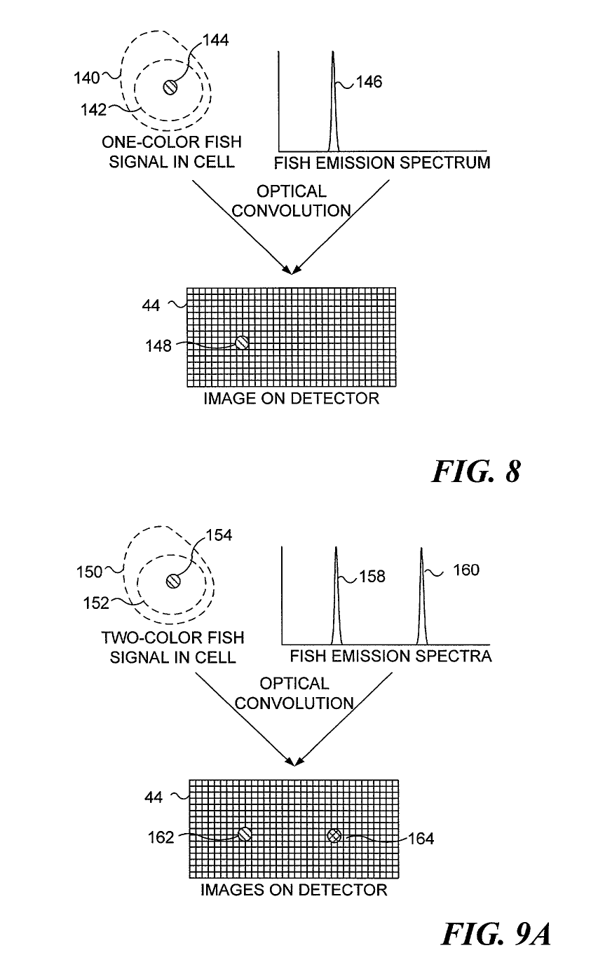

FIG. 8 illustrates an image on a detector produced by an embodiment of the present invention with a spectral resolution of approximately 10 nm per pixel and a spatial resolution of approximately 0.5 microns. In the following discussion of FIGS. 8 through 10, the operation of the present invention is directed toward the identification of multiplexed and non-multiplexed FISH probes bound to specific DNA with cells. FIG. 8 illustrates how the present invention is used to image a cell 140 having a nucleus 142 in which is disposed one non-multiplexed FISH probe 144 having an emission spectrum 146. Emission spectrum 146 of FISH probe 144 is approximately 10 nm in width and would be produced, for example, by “quantum dots” or a narrow-band fluorescent dye. The optical convolution of the narrow bandwidth spectrum results in minimal blurring of a FISH spot 148, enabling it to be readily resolved on detector 44.

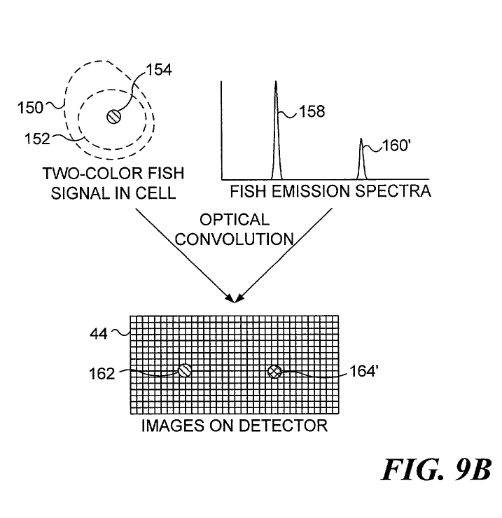

In FIG. 9A, a cell 150 is illustrated having a nucleus 152 in which is disposed a FISH probe 154 having two emission spectra. Each of the emission spectra of FISH probe 154 is relatively narrow, for example, corresponding to the emission spectra from quantum dots, as indicated by wavebands 158 and 160, and therefore, just as in FIG. 8, minimal blurring occurs in FISH spots 162 and 164 on detector 44. Furthermore, the spectral dispersion of the present invention, which maps wavelength into a lateral position on detector 44, produces a relatively wide physical displacement of FISH spots 162 and 164, despite the single source location of FISH probe 154 in the cell.

FIG. 9B illustrates the FISH emission spectra of FISH probe 154 when intensity is used to distinguish between different probes. As shown therein, a FISH emission spectrum 160′ is approximately one half the intensity of FISH emission spectrum 158. Corresponding FISH spots 162 and 164′ on TDI detector 44 will thus have two substantially different intensities, as well as being spectrally distinguishable. Accordingly, the relative intensities of the FISH spots produced on the detector can provide further information useful for identifying a FISH probe on an object being imaged.

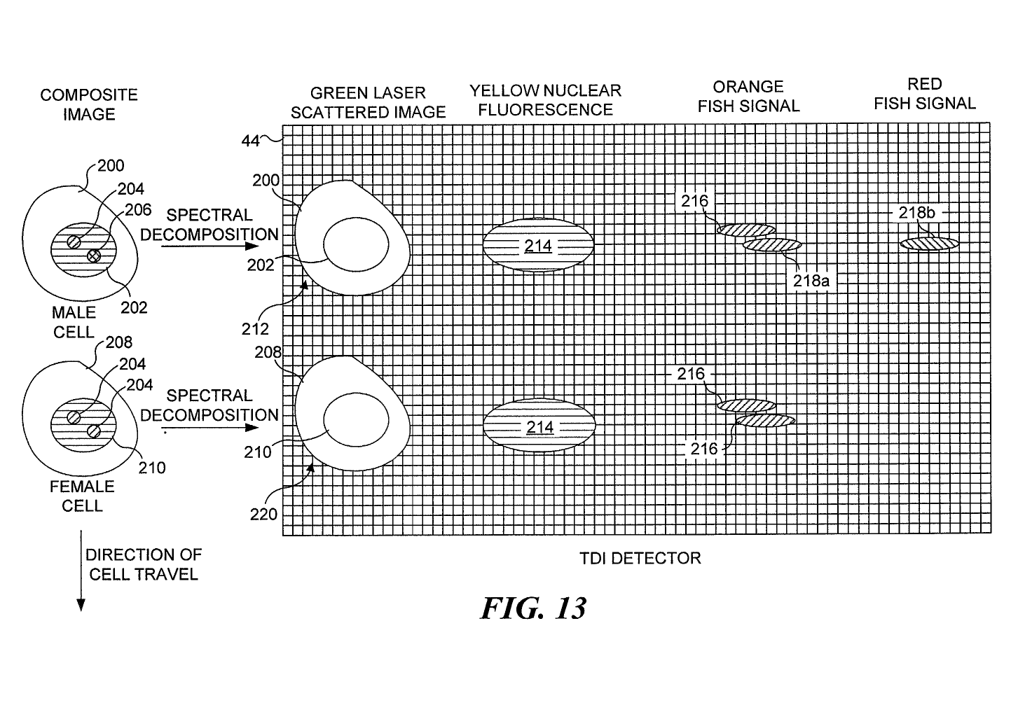

Taken together, FIGS. 8, 9A, and 9B illustrate how the present invention discriminates multiplexed and non-multiplexed FISH probes, thereby enabling the enumeration of numerous genetic traits. FIG. 13 illustrates how male and female cells 200 and 208, respectively, and FISH spots contained therein are imaged upon a pixilated detector in the present invention. Light of shorter wavelength, such as that producing a green laser scattered image 212, will be focussed on the left side of TDI detector 44. Light of slightly longer wavelength, such as a yellow nuclear fluorescence 214 from cell nuclei 202 or 210, will be laterally offset to the right. Light of still longer wavelengths, such as an orange FISH signal fluorescence 216 from an X-chromosome FISH probe 204 and a red FISH signal fluorescence 218 from a Y-chromosome FISH probe 206, will be focussed progressively farther to the right on the detector. In this manner, different components of a cell that fluoresce at different wavelengths will be focussed at different locations on the detector, while preserving the spatial information of those components. Each component image may be broadened laterally due to the width of its associated fluorescence emission spectrum. However, this broadening can be corrected based upon a priori knowledge of the emission spectra. Deconvolution of the emission spectrum from the broadened component image will yield an undistorted component image. Further, since the spectral dispersion characteristics of the spectral dispersing element are known, the lateral offsets of the different color component images can be corrected to reconstruct an accurate image of the cell.

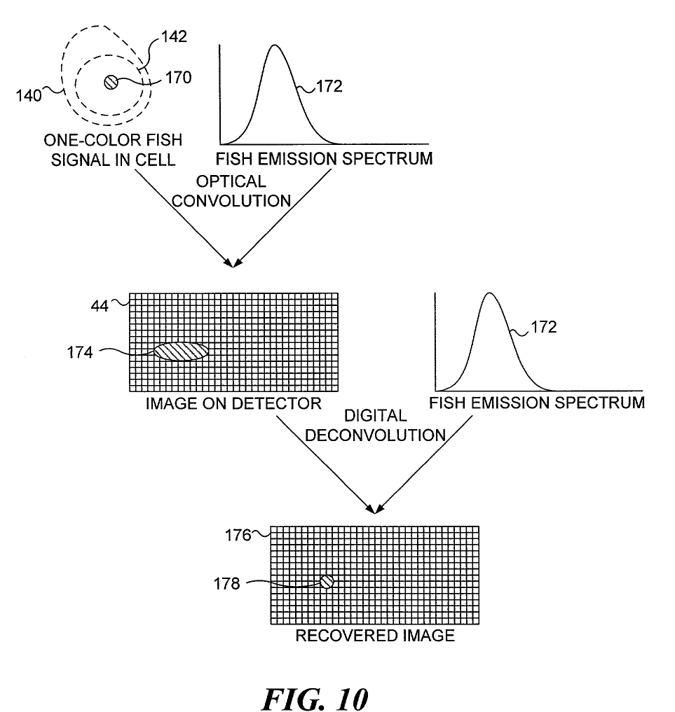

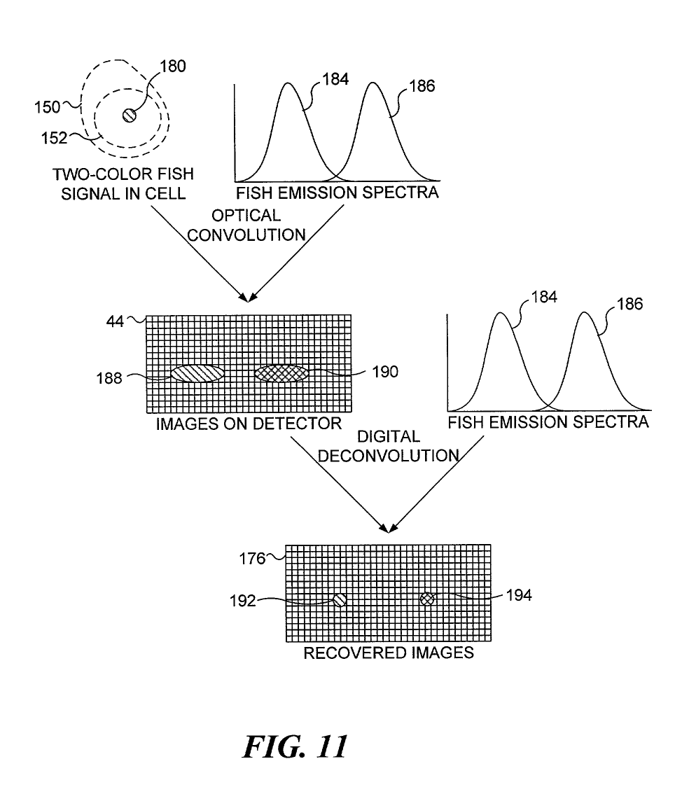

FIGS. 10 and 11 illustrate that the present invention can also be used with light of wider spectral bandwidth if an additional signal processing step is performed to correct for lateral blurring due to the wide emission spectra. In FIG. 10, a cell 140 having a nucleus 142 with a FISH probe 170 disposed in the nucleus is shown. FISH probe 170 is characterized by producing a relatively wide emission spectrum 172. When optically convolved by the spectral dispersion provided by the present invention, a FISH spot 174 is produced on detector 44, but the image is laterally blurred across the detector, as a result of the relatively wide emission spectrum. To more clearly resolve the separation of the FISH spot from probe 174, a deconvolution of the signal produced by detector 44 with the known FISH emission spectrum is carried out, producing an accurate FISH spot representation 178 on a display 176. The deconvolution step enhances the ability to enumerate multiple FISH spots within the cell in accord with the present invention.

In FIG. 11, a FISH probe 180 in a nucleus 152 of a cell 150 emits two relatively wide emission spectra 184 and 186. These relatively wide spectra produce corresponding laterally blurred FISH spots 188 and 190 on detector 44. Applying the deconvolution step, as noted above, produces accurate FISH spot representations 192 and 194 of the two spectra on display 176, as shown in the Figure.

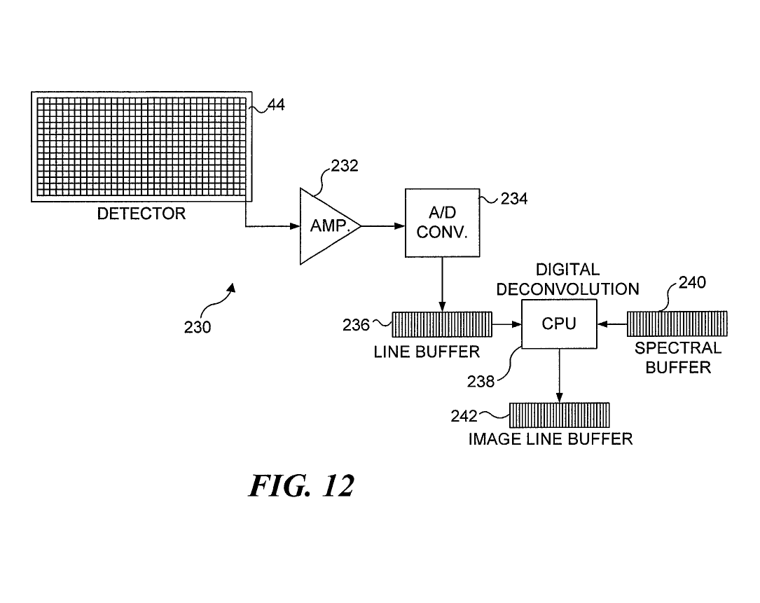

A system 230 for analyzing the signal produced by detector 44 and for performing the deconvolution steps described above is illustrated in FIG. 12. In FIG. 12, the signal from detector 44 is applied to an amplifier 232, which buffers the signal and amplifies it to achieve a level required by an analog-to-digital (A-D) converter 234. This A-D converter converts the analog signal from amplifier 232 into a digital signal that is input into a line buffer 236. Line buffer 236 temporarily stores the digital signal until it can be processed by a central processing unit (CPU) 238. To carry out the deconvolution noted above, a spectral buffer 240 is loaded with the known emission spectrum for each of the FISH probes being used so that their emission spectra can be deconvolved with the signal stored in line buffer 236. CPU 238 is a high speed processor programmed to carry out the deconvolution and other analysis procedures, enabling the identification of desired characteristics or parameters of the object being imaged. The output from CPU 238 is temporarily stored in an image line buffer 242 that enables the image to be displayed or otherwise recorded for later analysis.

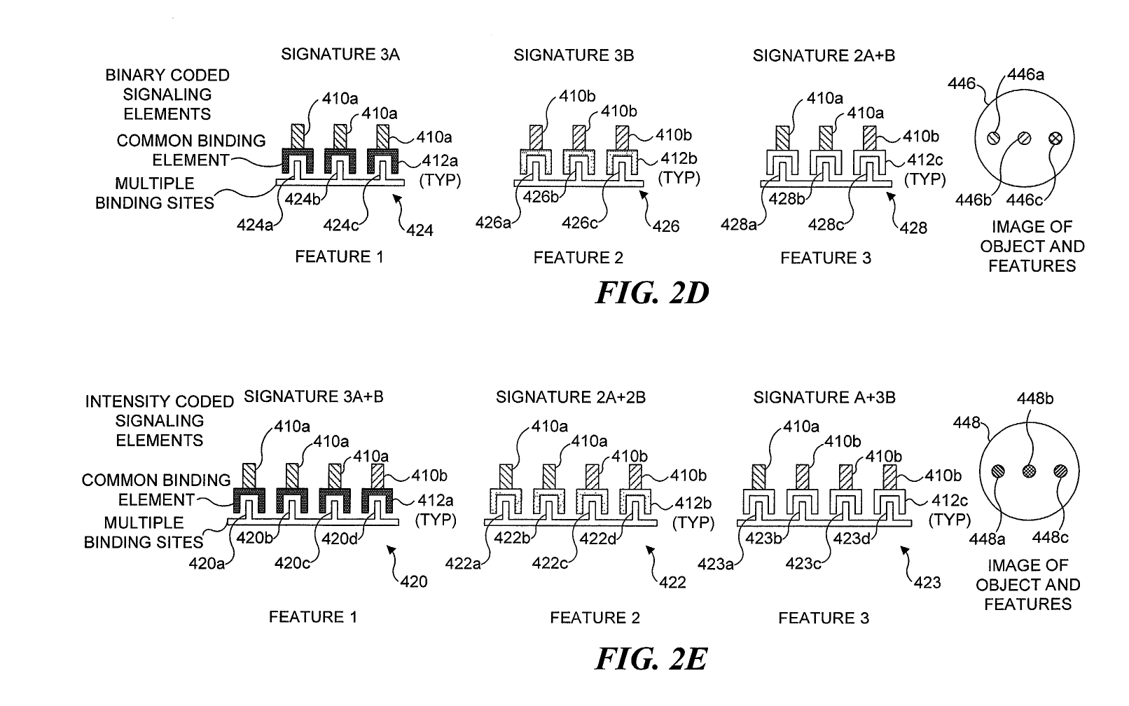

Those skilled in the art will appreciate that the present invention is intended for the discrimination of multiplexed probes. FIG. 3 illustrates the results of processing the imagery on the detector in a multiplexed probe scenario, in which the emission spectra from each probe have been deconvolved. In this Figure, an image 300′ of a cell 300 having a nucleus 302 that contains non-multiplexed probes 304, 306, 308, and 310 so that each probe generates an image 310′, 308′, 304′ and 306′, respectively, in only one color zone (blue, green, yellow, and red) on the detector. (Note that the relative positions of the other probes not imaged are shown as dotted line, unfilled circles in each of the other color zones.) An image 302′ of the nucleus appears in an indigo color zone.

A cell 316 having a nucleus 318 contains probes 320, 322, 324, and 326, which are multiplexed using four colors in the lower portion of FIG. 3. An image 318′ of the nucleus is included in the indigo color zone. Each of the multiplexed probes generates imagery in more than one color zone on the detector. Thus, in the blue color zone, images 320b and 326b are formed for probes 320 and 326, respectively; in the green color zone, images 320g and 322g are formed for probes 320 and 322; in the yellow color zone, images 320y and 324y are formed for probes 320 and 324; and in the red color zone, images 320r, 322r, and 326r are formed for probes 320, 322, and 326, respectively. (The relative locations of the other probes not imaged in each color zone are again indicated by dotted line, unfilled circles.) FIG. 3, which is discussed in greater detail below, also illustrates the use of the non-convolving spectral decomposition and imaging system discussed in the next section.

### Spectral Decomposition and Imaging

In one embodiment of the present invention, a spectral dispersing component having characteristics that ensure no distortion or convolution of the image occurs due to the emission bandwidth is employed, and as a result, a deconvolution is not needed to correct the image. A detailed disclosure of the spectral decomposition and imaging system is included in U.S. Pat. No. 6,211,955, entitled “Imaging and Analyzing Parameters of Small Moving Objects Such as Cells,” filed on Mar. 29, 2000, the disclosure and drawings of which are hereby specifically incorporated herein by reference.

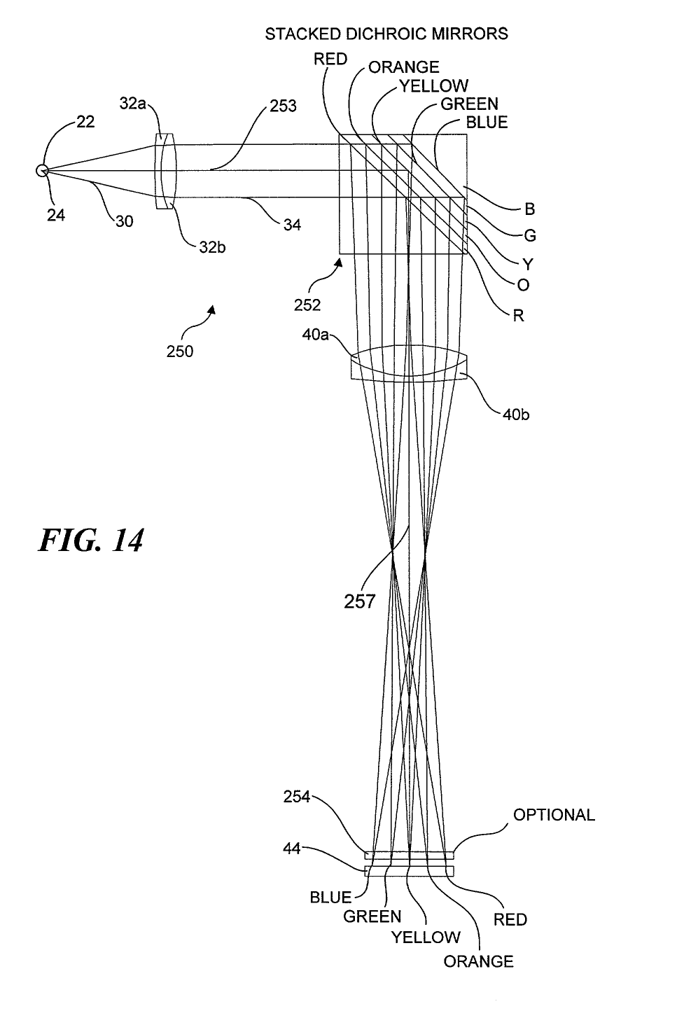

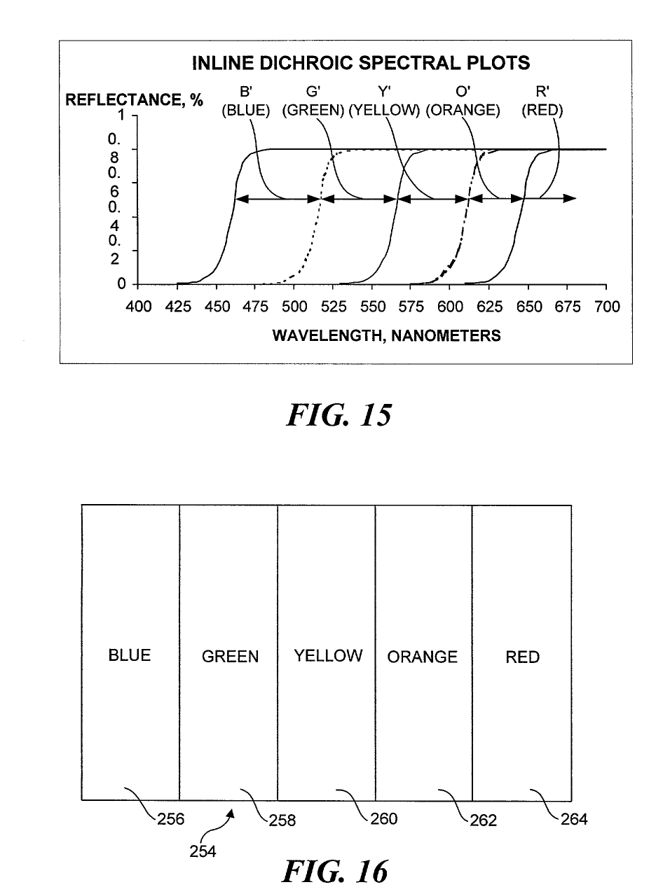

One preferred spectral dispersing component 250, which is illustrated in FIG. 14, comprises a plurality of dichroic beam splitters, such as dichroic mirrors, which are arranged to reflect light within different predefined bandwidths at different predefined angles. Unlike a prism, where light of different wavelengths leave the prism at different angles, all light within a predefined bandwidth incident on a dichroic beam splitter 252 at a common angle is reflected by the dichroic beam splitter at the same angle. Consequently, there is no convolution between the emission spectrum of the light leaving the object and the image of that object. When using such a spectral dispersing component, light of a first spectral bandwidth is reflected from the first dichroic beam splitter toward detector 44 at a predefined nominal angle. Light of a second spectral bandwidth passes through the first dichroic beam splitter to the next dichroic beam splitter and is reflected therefrom toward detector 44 at a different predefined nominal angle. Light of a third spectral bandwidth passes through the first and second dichroic beam splitters to a third dichroic beam splitter and is reflected therefrom at a third predefined nominal angle. Each angle of reflection is relative to an axis 257. The dichroic beam splitters are selected to cover the desired light spectrum with the appropriate spectral passbands. FIG. 15 illustrates the transmission characteristics of the dichroic filters used in the embodiment of FIG. 14. The angle of each dichroic beam splitter is set such that light reflected from it within the corresponding spectral bandwidth for the dichroic beam splitter is focussed onto a different region of the detector. Since the present invention uses a narrow field angle in object space along axis 257, perpendicular to the axis of motion, many different spectral bandwidths can be simultaneously imaged onto a single detector. In this manner, each region on the detector may cover a different spectral bandwidth, while light is collected over the same field angle in object space.

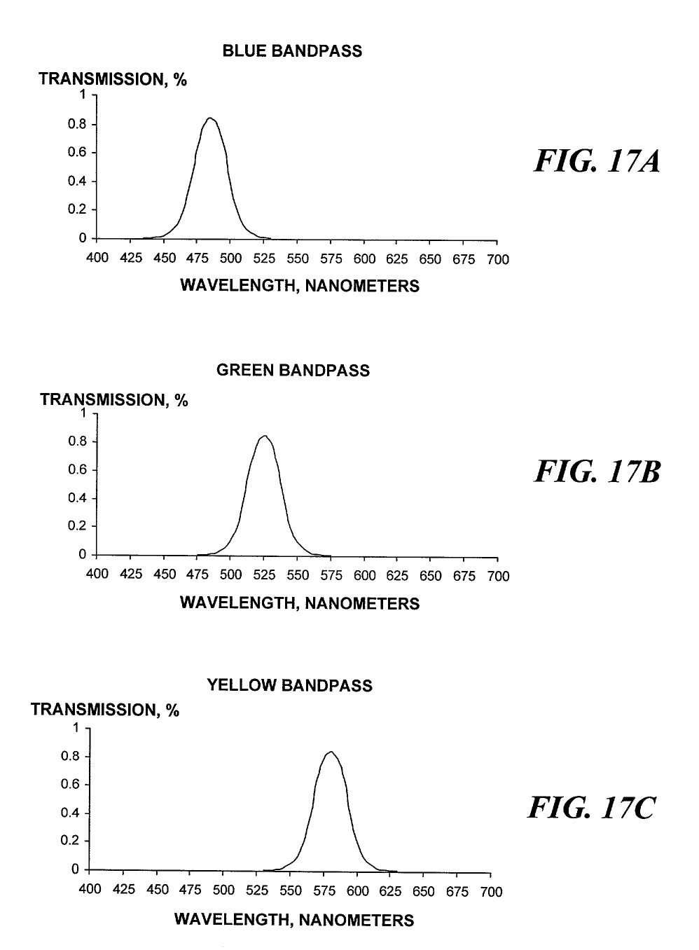

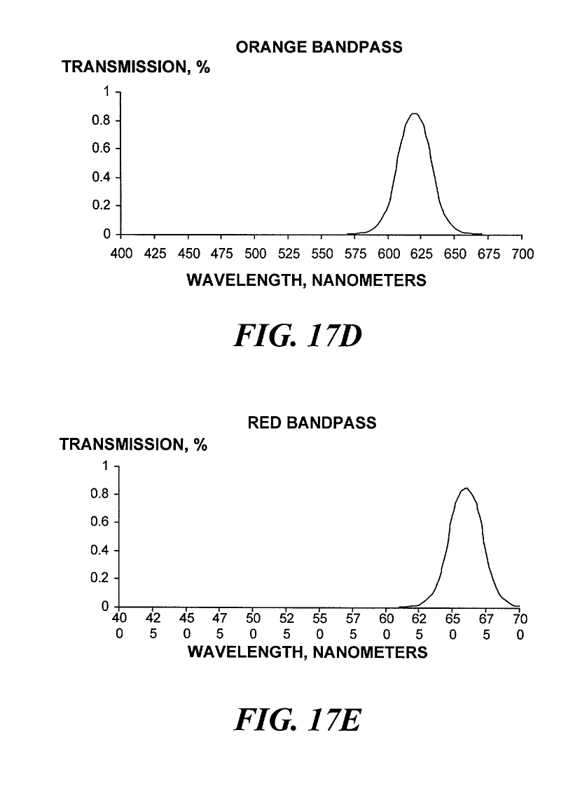

Depending on the amount of out-of-band rejection required, a bandpass filter 254 is optionally placed in front of detector 44. FIG. 16 illustrates the construction of a bandpass filter that may be placed immediately adjacent to the detector in the present invention. As shown in FIG. 16, the bandpass filter comprises a plurality of narrow spectral filters 256, 258, 260, 262, and 264 that are placed side-by-side to cover regions of the detector in correspondence with the spectral information to be imaged in those regions. FIGS. 17A-17E illustrate the spectral bandpass characteristics for each zone of the bandpass filter shown in FIG. 16. Since the position of each spectral bandwidth region is predefined, and since the present invention maintains the spatial integrity of the object, a full color, high spectral resolution representation of the object is generated from the spectral information imaged onto the detector.

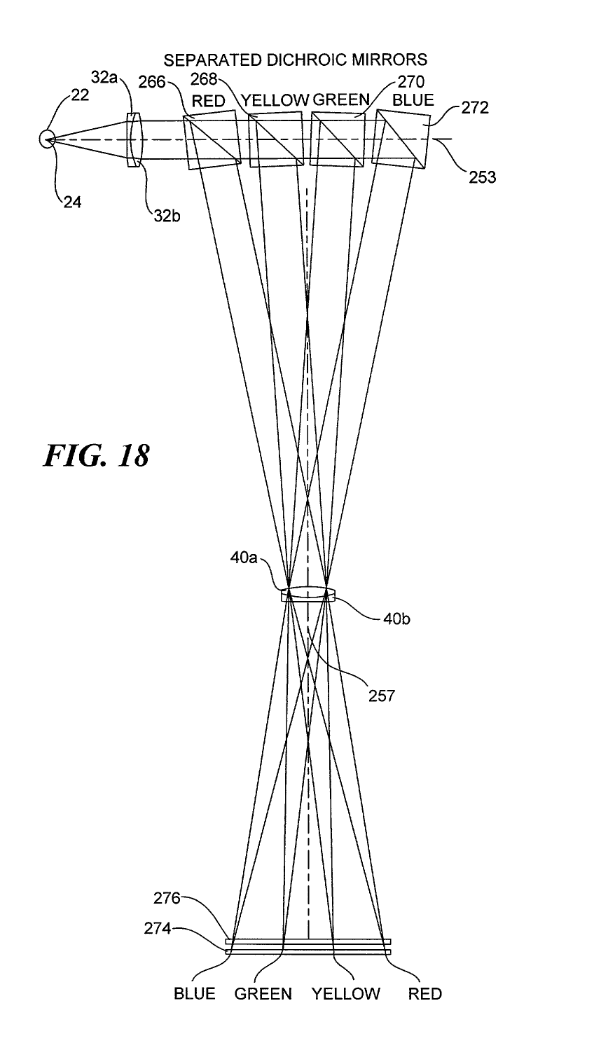

FIG. 18 illustrates another embodiment of the spectral dispersion component in which light does not pass through another dichroic filter after reflection from a dichroic filter component. In this embodiment, a plurality of cube dichroic filters, including a red cube filter 266, a yellow cube filter 268, a green cube filter 270, and a blue cube filter 272 are spaced apart sufficiently to ensure that light does not pass through any of the cube filters more than once. As with the embodiment shown in FIG. 14, the cube dichroic filters are oriented at appropriate angles to image light within a predefined bandwidth to distinct regions on a detector 274. As the light is reflected from each of cube dichroic filters 266, 268, 270 and 272, it is directed toward imaging lenses 40a and 40b, and different bandpass portions of the light are focussed upon corresponding red, yellow, green, and blue light receiving segments or regions defined on a light receiving surface of detector 274. If desired, an optional detector filter assembly 276 of similar construction to bandpass filter 254 (but without the orange spectral region) may be used to increase the rejection of out-of-band signals. It should be apparent to those of ordinary skill in the art that separate spaced-apart plate, or pellical beam splitters could alternatively be used in this application instead of the cube filters. In the embodiment illustrated in FIG. 18, imaging lenses 40a and 40b must be placed a sufficient distance away from the plurality of cube filters to minimize the clear aperture requirement for lenses 40a and 40b. Those skilled in the art will appreciate that the clear aperture in the plane orthogonal to the page can increase as the distance between the lenses and plurality cube filters increases. Therefore, the placement of lenses 40a and 40b must be chosen to appropriately accommodate the clear aperture in both planes.

The foregoing descriptions of these preferred embodiments of spectral dispersion components illustrate the use of four and five color systems. Those skilled in the art will appreciate that a spectral dispersing component with more or fewer filters may be used in these configurations in order to construct a system covering a wider or a narrower spectral region, or to provide different passbands within a given spectral region. Likewise, those skilled in the art will appreciate that the spectral resolution of the present invention may be increased or decreased by appropriately choosing the number and spectral characteristics of the dichroic and/or bandpass filters that are used. Furthermore, the angles or orientation of the filters may be adjusted to direct light of a given bandwidth onto any desired point on the detector. The use of these preferred embodiments for imaging light from an object are not limited to objects in flow, but may also be applied to imaging objects on substrates as long as the FOV is sufficiently narrow in the axis substantially orthogonal to the plane of spectral decomposition to prevent crosstalk between the decomposed images.

FIG. 3 illustrates the images projected onto a detector for the present spectral decomposition embodiment in the case where two cells are in view. In this illustration, each cell 300 and 316 has a series of four unique multiplexed probes visible, in which the probes are constructed of up to four distinct fluorochromes. There is no spreading of the probe images on the detector, as occurs in the prism-based spectral decomposition embodiment. Cell 300 utilizes one type of signaling element per probe, while cell 316 uses a multiplexed combination of up to four signaling elements per probe.

### Another Embodiment for Spectral Decomposition and Imaging

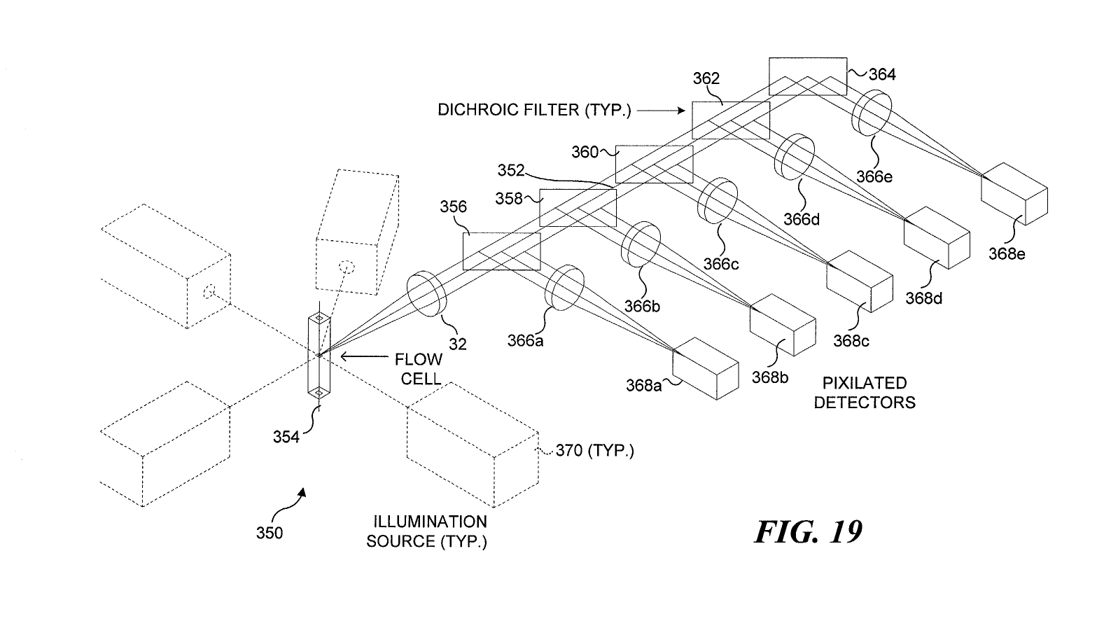

An embodiment 350 of an imaging system, which is illustrated in FIG. 19, is similar to the embodiments described above in that no convolution of the emission spectra with the image occurs as a result of the spectral decomposition process. One or more illumination sources 370 are optionally used to illuminate the objects. Spectral decomposition occurs in an axis 352 that is perpendicular to a flow 354 through the use of dichroic filters 356, 358, 360, 362, and 364, generally as previously described above. However, in this embodiment, separate imaging lenses 366a, 366b, 366c, 366d, and 366e and separate pixilated detectors 368a, 368b, 368c, 368d, and 368e are used for each spectral region. In this configuration, each detector may have a very narrow aspect ratio, and very few pixels are required in the axis perpendicular to flow, since the field angle in that axis is approximately 25 arc minutes. In the case where a six-color version of this embodiment is employed, the images on a single detector would appear like the images seen on one zone of the detector illustrated in FIG. 3. For example, the images seen on the detector configured to receive light in the red part of the spectrum would appear like the right-most zone or red color zone of FIG. 3. Since the total number of pixels in each pixilated detector is low, these detectors may operate at very high speeds.

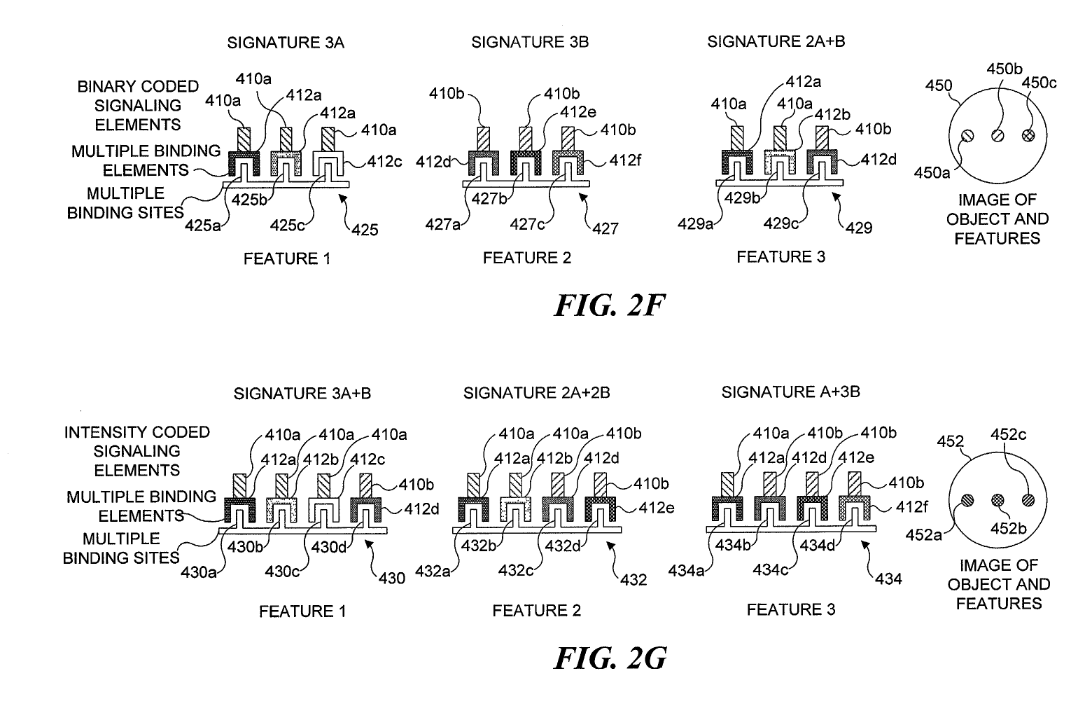

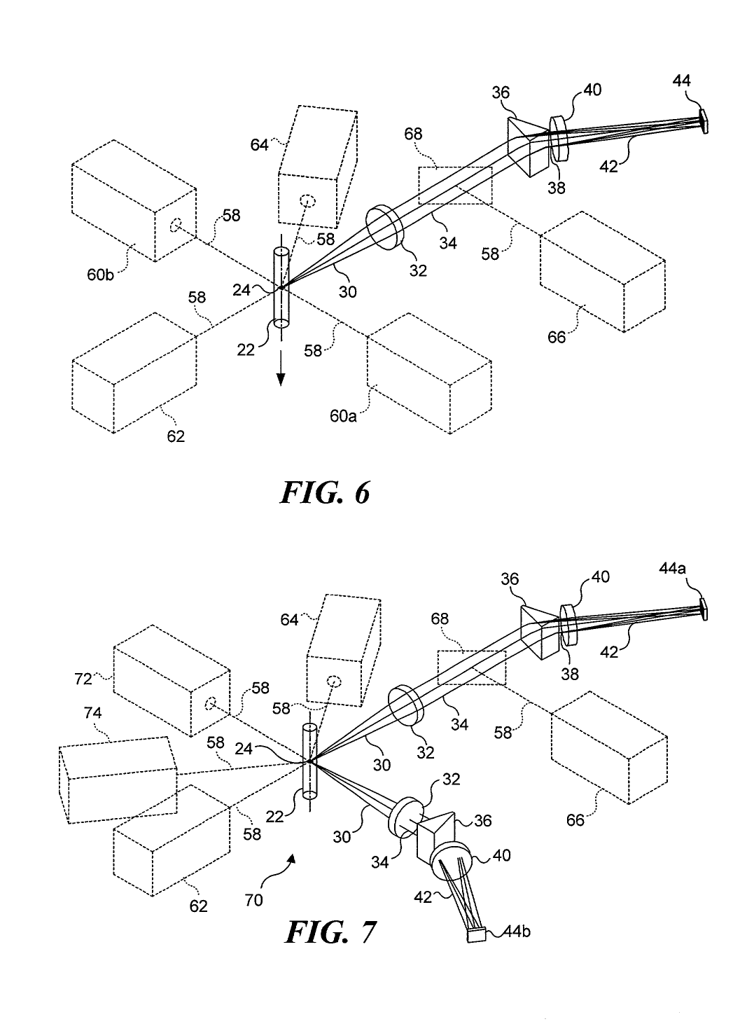

As disclosed in the above-referenced U.S. Pat. No. 6,249,341 and as shown in FIGS. 4 and 4A, and in FIG. 7, multiple legs of the spectral decomposition and imaging system may be used to collect signals from multiple perspectives as cell (or other objects) 24 flow through a cuvette 23 having a square cross section. Two legs are shown in FIG. 4, one having a detector 44a and the other a detector 44b. However, if epi illumination is applied, as shown in FIGS. 6 and 7, four legs may be used to view the cells from each side of the cuvette. Light emitted from the objects may also be collected with objective lenses that can be optically coupled directly to the cuvette to improve the numeric aperture. In addition, each optical leg can image objects at multiple focal planes to improve focus on cells which may be defocused at a different focal plane.

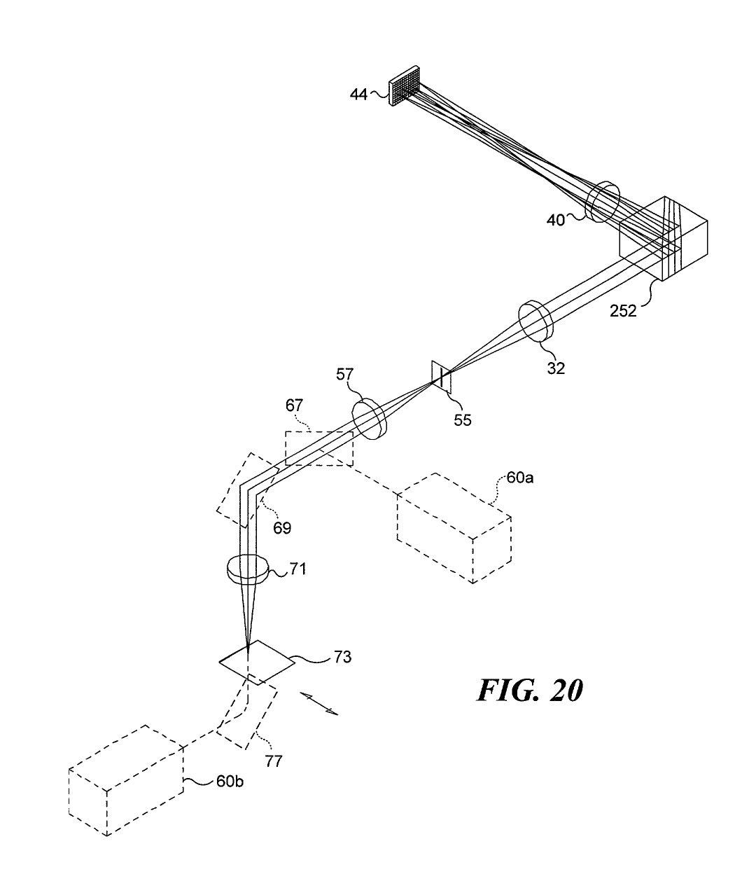

Although each of the previous four embodiments for spectral decomposition were described in the context of imaging objects in flow, those skilled in the art will appreciate that the same spectral decomposition systems can be applied to objects fixed to slides, microtiter plates, or other solid substrates. FIG. 20 illustrates an embodiment in which motion of a substrate 73 is generally parallel or aligned with an axis of spectral decomposition provided by dichroic beam splitter 252. An optional epi illuminator 60a, which may comprise a laser or other type of illumination source, can be used to illuminate objects carried on substrate 73, while there is relative movement between the substrate and the imaging system in the direction of the double-headed arrow. Optionally, another illuminator 60b is provided to provide bright field illumination of the objects on the substrate with light reflected from a reflective surface 77. Light from the objects on substrate 73 passes through a lens 71, is reflected from a reflective surface 69, passes through a dichroic (or partially reflective) mirror 67 and is focussed on a slit 55 by a lens 57. Collection lens 32 collimates the light from the slit and directs the light onto dichroic beam splitter 252, which spectrally disperses the light passing through lens 40 and onto different regions of detector 44, such as is shown in FIG. 21.

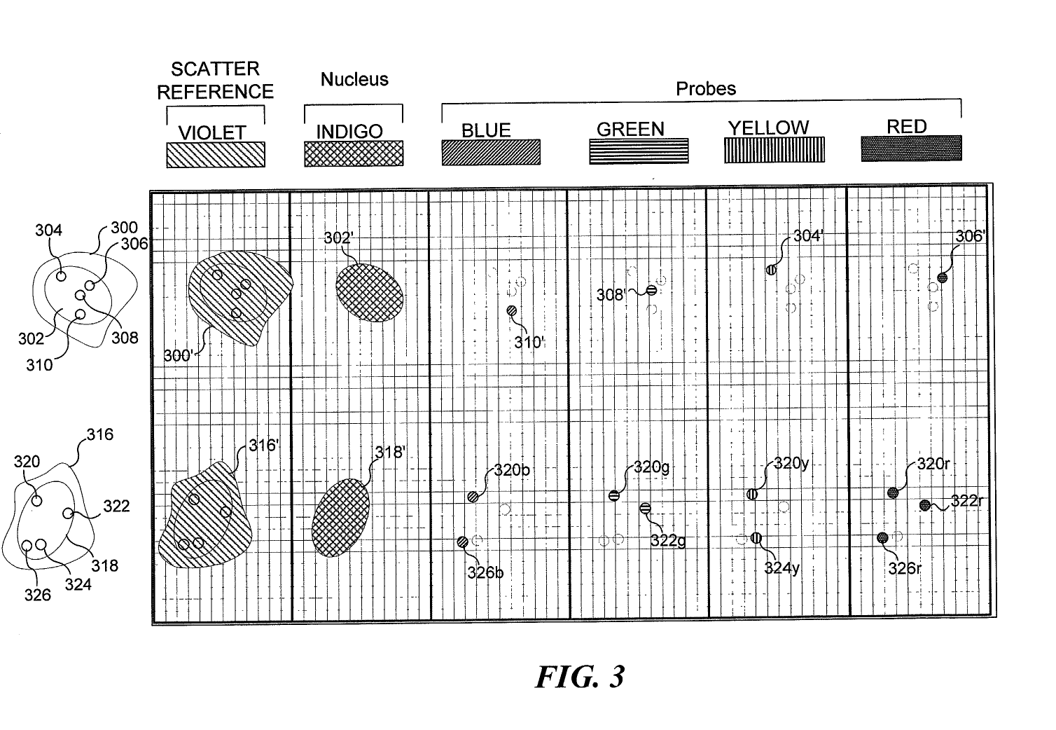

FIG. 5 also illustrates the application of the second spectral decomposition embodiment employed in an optical system to collect imagery from a solid substrate 73′, which may be transparent, translucent, or opaque. In this embodiment, a FOV 75 is either illuminated, for example, with light from epi illuminator 60. If used, light from the illuminator is directed by dichroic (or partially reflective mirror) 67 and by reflective surface 69 through a focussing lens 71 toward substrate 73′. Light from the substrate following this same path, is transmitted through the dichroic (or partially reflective) mirror and through lens 57, which focusses the light on slit 55. Light passing through the slit is collimated by collection lens 32 and directed to dichroic beam splitter 252, which spectral disperses the light onto different regions of detector 44. Those skilled in the art will also appreciate that where the fourth embodiment of the spectral decomposition system is employed, the FOV may be increased substantially in the axis perpendicular to movement to further increase throughput.

### First Embodiment for Pixilated Detection

The first pixilated detection embodiment employs frame-based charge coupled device (CCD) image collection, in which a CCD detector views cells in flow in a freeze frame fashion. This method requires the integration time to be very short to prevent blurring. A short integration time is achieved either with a strobed light source, or a continuous light source combined with a shuttered or gated detector. In either case, the short integration time reduces the signal-to-noise ratio and the ultimate sensitivity of the approach with fluorescence signals. Further, frame-based cameras require time to transfer data out of the camera, during which no images are acquired, and cells of interest can escape detection. However, these types of detectors are readily available, inexpensive, and do not require an accurate knowledge of the velocity of the cells in flow.

### Second Embodiment for Pixilated Detection

A second embodiment of flow imaging for pixilated detection also employs frame-based CCD image collection, but does not rely on strobed illumination or shuttered detection to freeze image motion. Instead, a rotating or oscillating mirror is used to compensate for object motion to produce a still image on the detector. This embodiment may employ continuous illumination, thereby achieving higher levels of sensitivity than strobed systems when analyzing fluorescence. However, this embodiment requires both an accurate measurement of object velocity and a very stable fluid pumping system, since the inertia of the mirror prevents compensation for rapid changes in object velocity.

### Third Embodiment for Pixilated Detection

A third embodiment for pixilated detection uses TDI CCD image collection. In TDI detection, the electronic signal produced within the detector by an incident image is moved down the detector in synchrony with the motion of the image. In this manner, signal integration times can be increased over conventional frame imaging modes by a factor exceeding 1000 fold. FIGS. 3, 13, and 21 illustrate such a detector and shows the spectral zones in which the detector is divided.

### Object Illumination

As shown in FIGS. 6 and 7, several different illumination systems may be employed to illuminate the cells in flow. A standard approach involves illuminating the cells in flow with a laser path oriented orthogonal to the spectral decomposition and imaging system. Alternative modes of illumination, such as those shown and FIGS. 6 and 7 and disclosed in above-referenced U.S. Pat. No. 6,249,341, allow for the generation of bright field, dark field, phase contrast, fluorescence and epi-fluorescence imagery. The above-referenced commonly assigned U.S. patent application Ser. No. 09/689,172, entitled “Multi-Pass Cavity for Illumination and Excitation of Moving Objects,” filed on Oct. 11, 2000, discloses a method for illumination in which the number of photons incident on the cells may increased by a factor of 10 or more.

The design of the illumination system also allows the use of pulsed lasers or other strobed sources for high sensitivity fluorescence measurement without any need to strobe in synchrony with object flow. Solid state pulsed lasers can be orders of magnitude more efficient than gas lasers of similar average power and their high peak energy allows for efficient conversion of visible output to ultraviolet. The high aspect ratio also allows for highly efficient coupling of linear array diode illumination into the cuvette.

### Cell Velocity Measurement

For the second and third pixilated detection embodiments wherein an accurate knowledge of the cell velocity is required, a frequency domain velocity measurement (FDVM) technique as disclosed in U.S. Provisional Patent Application No. 60/228,076, filed on Aug. 25, 2000 and entitled “Frequency Domain Object Velocity Measurement,” can be employed. In FDVM, a large FOV is imaged onto a ruling of opaque and transparent bars. Motion of the objects within the FOV causes modulation of their intensity as they pass across the ruling. The modulation frequency is proportional to the velocity of the objects and can be determined using Fast Fourier Transform analysis.

Velocity can also be determined using two detectors in a conventional time-of-flight measurement scheme, though with very restricted throughput. As described in U.S. Provisional Patent Application Serial No. 60/228,076, time-of-flight measurements become more complex when throughput increases and the times of flight of multiple objects are measured simultaneously. Such systems can fail when correlation is lost between the entry and exit times of the objects in view. The time-of-flight system preferably used relies on an improved scheme wherein the waveforms produced by the entry and exit detectors are cross-correlated to detect phase changes that are indicative of changes in velocity. When the present invention is applied to objects on a solid substrate and when imaging in TDI mode, an encoder, laser interferometer, or other means may be used to determine the object velocity in order to synchronize the TDI detector.

Although the present invention has been described in connection with the preferred form of practicing it, those of ordinary skill in the art will understand that many modifications can be made thereto within the scope of the claims that follow. Accordingly, it is not intended that the scope of the invention in any way be limited by the above description, but instead be determined entirely by reference to the claims that follow.

The invention in which an exclusive right is claimed is defined by the following:

1. In a set of labeled probes useful for optically marking a feature of an object, said set of labeled probes including at least one labeled probe that binds to said feature and generates a signal that can be used to identify said feature, any combination of labeled probes useful for marking the feature being uniquely optically discriminable, each labeled probe in said set of labeled probes comprising:
(a) a binding element selectively binding to at least a portion of said feature; and
(b) at least one signaling component coupled to the binding element, so that:
(i) if only one binding element is bound to said feature, a plurality of signaling components that are optically discriminable, but need not be spatially discriminable are coupled to said binding element; and
(ii) if a plurality of binding elements are bound to said feature, at least one signaling component is coupled to each binding element to thereby bind a plurality of signaling components to the feature, said signaling components being optically discriminable, but not required to be spatially discriminable, when providing a unique optical signature that optically marks the feature.

2. The set of labeled probes of claim 1, wherein the unique optical signature of the labeled probes of said set that marks the feature comprises at least one of:
(a) a spectral signature for all signaling components coupled to said at least one binding element that is selectively bound to the feature; and
(b) an intensity of a waveband of light produced by the signaling components coupled to said at least one binding element that is selectively bound to the feature.

3. The set of labeled probes of claim 1, wherein when only one binding element is bound to said feature, said plurality of signaling components comprises at least two optically distinguishable signaling components.

4. The set of labeled probes of claim 1, wherein when only one binding element is bound to said feature, said plurality of signaling components comprises identical signaling components, enabling said feature to be optically distinguished based on an intensity of the signal produced by the signaling components that are coupled to said feature by the binding element.

5. The set of labeled probes of claim 1, wherein when a plurality of binding elements are bound to said feature, each of said at least one signaling component coupled to each binding element is identical, enabling said feature to be optically distinguished by an intensity of the signal produced by the signaling components that are coupled with said feature by the plurality of binding elements.

6. The set of labeled probes of claim 1, wherein when a plurality of binding elements are bound to said feature, each of said at least one signaling component coupled to each binding element is selected from at least two different types of signaling components, such that said feature is marked by the unique optical signature that comprises a combination of the optical signatures of said at least two different signaling components.

7. The set of labeled probes of Claim 1, wherein when a plurality of binding elements are bound to said feature, each of said plurality of binding elements is identical.

8. The set of labeled probes of claim 1, wherein when a plurality of binding elements are bound to said feature, each of said plurality of binding elements is different.

9. A set of labeled probes useful for indicating one or more features of an object, comprising:
(a) at least one binding element that selectively binds to a binding site of a feature; and
(b) a plurality of signaling components that are optically discriminable, but not required to be spatially discriminable and which together produce a signal indicative of the feature, said at least one binding element coupling to:
(i) two or more signaling components if the feature has only a single binding site to which said at least one binding element is bound; and,
(ii) at least one signaling component if the feature has a plurality of binding sites to which said at least one binding element is bound, so that at least a pair of the signaling components are coupled to the feature, providing a distinctive indication of the feature on the object.

10. The set of claim 9, wherein the plurality of signaling components are identical, enabling a feature to be optically distinguished based upon an intensity of the signal produced by the signaling components coupled to the feature.

11. The set of claim 9, wherein the plurality of signaling components comprise at least two different types of signaling components.

12. The set of claim 9, wherein when said at least one binding element couples to two or more signaling components, each signaling component coupled to the same binding element is identical, enabling a feature to be optically distinguished based upon an intensity of the signal produced by the signaling components coupled to said feature.

13. The set of claim 9, wherein when said at least one binding element couples a plurality of signaling components to a feature, said plurality of signaling components comprises at least two different types of signaling components.

14. The set of claim 9, wherein when said at least one binding element couples at least one signaling component to a feature, said specific feature is optically indicated based on an intensity of the signal produced by said at least one signaling component that is coupled to said feature.

15. The set of claim 9, wherein said at least one binding element comprises a plurality of identical binding elements.

16. The set of claim 9, wherein said at least one binding element comprises a plurality of binding elements, not all of which are identical.

17. A set of labeled probes useful for indicating different features of an object, comprising:
(a) at least one binding element that binds to at least one of the different features; and
(b) at least one signaling component coupled to said at least one binding element and thus coupled to the feature to which said at least one binding element is bound, producing a uniquely discriminable optical signal, so that at least two different labeled probes of the set that bind to different features share at least one different signaling component in common, the signaling components bound to a feature not being required to be spatially discriminable.

18. The set of claim 17, wherein each labeled probe binding to the same feature comprises the same binding element.

19. The set of claim 17, wherein each labeled probe binding to the same feature comprises only one signaling component, such that at least two labeled probes binding to the same feature have different signaling components.

20. The set of claim 17, wherein at least two labeled probes binding to the same feature have different binding elements.

21. A set of labeled probes useful for indicating a presence of a plurality of different features of an object, said set of labeled probes comprising:
(a) a plurality of different binding elements, each different binding element selectively binding to at least a portion of a feature of the object; and
(b) a plurality of signaling components, each different signaling component having a unique spectral signature, a number of different signaling components in said set being fewer than a number of the different binding elements in said set, such that at least two different labeled probes in the set that are capable of binding to different features share at least one different signaling component in common, labeled probes that are bound to a feature being optically discriminable, but not being required to be spatially discriminable.

22. The set of claim 21, wherein the labeled probes of said set indicate the presence of a specific feature based on at least one of:
(a) a spectral signature of each signaling component coupled to the specific feature by at least one binding element that selectively binds to the specific feature; and
(b) an intensity of a waveband of light produced by each signaling component that is coupled to the specific feature by at least one binding element that selectively binds to the specific feature.

23. A set of probes useful for marking a plurality of different features associated with an object as a function of multiplexed optical signals that uniquely indicate each different feature, said set comprising:
a plurality of different labeled probes, each different labeled probe having a uniquely optically identifiable characteristic, a binding element that selectively binds to at least a portion of at least one feature of said plurality of different features, and at least one signaling component coupled to a feature by said binding element, each labeled probe providing a multiplexed optical signal enabling different features associated with an object to me identified, said multiplexed optical signal comprising a combination of optical signals produced by each signaling component that is coupled to the feature, said multiplexed optical signal being optically discriminable, but not required to be spatially discriminable, wherein the plurality of labeled probes comprises at least:
(a) a first labeled probe selected to bind to at least a portion of a first feature;
(b) a second labeled probe selected to bind to at least a portion of a second feature that is different than the first feature, such that said first and second labeled probes include at least one signaling component in common in the multiplexed optical signal that each produces to enable identification of the first and the second features.

24. A system for marking and identifying at least one feature associated with an object, said system comprising:
(a) a set of labeled probes useful for optically marking a feature of an object, said set of labeled probes including at least one labeled probe that binds to said feature and provides an optical signal that can be used to identify said feature, each different labeled probe in the set being uniquely optically discriminable and comprising a binding element selectively binding to at least a portion of said feature, and at least one signaling component coupled to the binding element, so that;
(i) if only one binding element is bound to said feature, a plurality of signaling components that are optically discriminable, but not spatially discriminable are coupled to said binding element; and
(ii) if a plurality of binding elements are bound to said feature, at least one signaling component is coupled to each binding element to thereby bind a plurality of signaling components to the feature, said signaling components being optically discriminable, but not spatially discriminable by the system, when providing a unique optical signature that marks the feature; and
(b) a light detector that detects the labeled probes bound to said at least one feature associated with the object, based upon the unique optical signature provided by the signaling components that are coupled to said at least one feature.

25. The system of claim 24, wherein the light detector comprises:
(a) a collection lens disposed so that light traveling from the object passes through the collection lens and is focused along a collection path;
(b) a dispersing component that receives the light from the collection lens and disperses the light into a plurality of light beams, as a function of a plurality of different discriminable optical characteristics of the light, said plurality of different discriminable optical characteristics being indicative of each different signaling component that is coupled to said at least one feature;
(c) at least one pixilated detector;
(d) an imaging lens that focuses each of the plurality of light beams on said at least one pixilated detector, producing a respective image corresponding to each of the plurality of light beams, said at least one pixilated detector providing an output signal for each respective image, each output signal indicating each different signaling component that is coupled to said at least one feature associated with the object; and
(e) a signal processor coupled to receive the output signals from said at least one pixilated detector, said signal processor processing the output signals to determine each labeled probe that is bound to said at least one feature associated with the object.

26. The system of claim 25, wherein said dispersing component comprises one of a dichroic filters and a prism.

27. The system of claim 25, wherein said at least one pixilated detector comprises a time delay integration (TDI) detector.

28. The system of claim 25, wherein said imaging lens focuses each one of said plurality of light beams onto a different region of said at least one pixilated detector.

29. The system of claim 25, further comprising at least one light source for illuminating the object.

30. The system of claim 24, wherein said light detector comprises:
(a) a collection lens disposed so that light traveling from the object passes through the collection lens and travels along a collection path;
(b) a plurality of light reflecting elements disposed in the collection path, each light reflecting element reflecting light of a different predefined characteristic, and passing light that does not have that predefined characteristic, the signaling components coupled to said at least one feature associated with the object determining the characteristics of light traveling along the collection path, each light reflecting element being positioned at a different location with respect to the collection path to reflect light of a specific predefined characteristic in a direction different from that of other light reflecting elements, each light reflecting element being positioned along an axis of said collection path, such that passing light not reflected by a preceding light reflecting element reaches a last light reflecting element;
(c) at least one pixilated detector disposed to receive light that has been reflected by each of the light reflecting elements, said at least one pixilated detector comprising a plurality of pixilated regions, each pixilated region producing an output signal indicating each different signaling component that is coupled to said at least one feature associated with the object; and
(d) a signal processor coupled to receive each output signal from said the plurality of regions, said signal processor processing each output signal to determine each labeled probe that is bound to said at least one feature associated with the object.

31. A system for marking and identifying one or more features of an object, said system comprising:
(a) a set of labeled probes useful for marking features of the object, each labeled probe of the set of labeled probes comprising:
(i) at least one binding element that binds to at least one feature; and
(ii) at least one signaling component coupled to said at least one binding element and thus coupled to the feature to which said at least one binding element is bound, producing a uniquely discriminable optical signal, so that at least two different labeled probes of the set that bind to different features share at least one different signaling component in common, the signaling components bound to a feature being optically discriminable, but not required to be spatially discriminable; and
(b) a cytometer for imaging the object and identifying each feature marked by at least one labeled probe of the set, by simultaneously detecting the uniquely discriminable optical signatures of any signaling components that are coupled to any feature of the object by said at least one binding element.

32. The system of claim 31, wherein said cytometer comprises:
(a) a collection lens disposed so that light traveling from the object passes through the collection lens and is focused along a collection path;
(b) a dispersing component that receives the light from the collection lens and disperses the light into a plurality of light beams, as a function of a plurality of different discriminable characteristics of the light, said plurality of different discriminable characteristics being indicative of the signaling components coupled to any features associated with the object;
(c) at least one pixilated detector;
(d) an imaging lens that focuses each of the plurality of light beams on said at least one pixilated detector, producing a respective image corresponding to each of the plurality of light beams, said at least one pixilated detector simultaneously providing an output signal for each respective image, each output signal indicating a different one of the plurality of signaling components bound to any features associated with the object; and
(e) a signal processor coupled to receive the output signals from said at least one pixilated detector, said signal processor processing the output signals to determine if the object has one or more features labeled with any of said plurality of labeled probes, to detect said one or more features.

33. The system of claim 31, wherein said cytometer comprises:
(a) a collection lens disposed so that light traveling from the object passes through the collection lens and travels along a collection path;
(b) a plurality of light reflecting elements disposed in the collection path, each light reflecting element reflecting light of a different predefined characteristic, and passing light that does not have that predefined characteristic, each signaling components coupled to a feature of the object determining the characteristics of light traveling along the collection path, each light reflecting element being positioned at a different location with respect to the collection path to reflect light of a specific predefined characteristic in a direction different from that of other light reflecting elements, each light reflecting element being positioned along an axis of said collection path, such that passing light not reflected by a preceding light reflecting element reaches a last light reflecting element;
(c) at least one pixilated detector disposed to receive light that has been reflected by each of the light reflecting elements, said at least one pixilated detector comprising a plurality of pixilated regions, each pixilated region producing an output signal that is indicative of a different uniquely optically discriminable characteristic of the signaling components and thus indicative of any labeled probe marking a feature on the object; and
(d) a signal processor coupled to receive the output signals from said the plurality of regions, said signal processor processing the output signals to determine if any feature is labeled with one of said plurality of labeled probes, and thus to identify any such feature.

34. A method for detecting a feature on an object using an imaging system, comprising the steps of:
(a) providing at least one labeled probe that selectively binds to said feature, wherein said at least one labeled probe comprises a binding element that selectively binds to at least a portion of said feature, and at least one optical signaling component;
(b) exposing said object to said at least one labeled probe under conditions that cause said at least one labeled probe to bind to at least a portion of said feature, if said feature is associated with said object, such that a plurality of optical signaling components become bound to said feature;
(c) collecting light from said object along a collection path;
(d) focusing the collected light to produce an image corresponding to the object, locations of labeled probes bound to a feature included in the image being optically discriminated but not spatially discriminated in the image;
(e) detecting the image to produce a signal indicative of each optical signaling component bound to the feature on the object;
(f) analyzing the signal to determine if a spectral component due to the each optical signaling component bound to said feature is present in the image, thereby establishing that the feature is associated with the object.

35. The method of claim 34, wherein the step of exposing said object to said at least one labeled probe comprises the step of exposing said object to a labeled probe that comprises said plurality of optical signaling components, thereby binding said plurality of optical signaling components to said feature.

36. The method of claim 35, wherein the step of exposing said object to a labeled probe comprises the step of exposing said object to a labeled probe that comprises a plurality of identical optical signaling components.

37. The method of claim 36, wherein the step of analyzing the signal comprises the step of determining if an intensity of a waveband of light indicative of a plurality of optical signaling components is present in the image.

38. The method of claim 35, wherein the step of exposing said object to a labeled probe comprises the step of exposing said object to a labeled probe that comprises a plurality of different optical signaling components.

39. The method of claim 38, wherein the step of analyzing the signal comprises the step of determining if a multiplex of a spectral signature for each of the plurality of different optical signaling components is present in the image.

40. The method of claim 35, wherein the step of exposing said object to a labeled probe that comprises the plurality of optical signaling components comprises the step of exposing said object to at least two labeled probes, each of which comprises a binding element that selectively binds to at least a portion of the feature, and each of which comprises at least one optical signaling component, thereby binding the plurality of optical signaling components to said feature.

41. The method of claim 34, further comprising the step of dispersing the light that is traveling along the collection path into a plurality of light beams, as a function of a plurality of different discriminable characteristics of the light; wherein:
(a) the step of focusing the collected light to produce an image corresponding to the object comprises the step of focusing each of the plurality of light beams to produce a respective image corresponding to that light beam, thereby generating a plurality of images;
(b) the step of detecting the image comprises the step of responding to each of the plurality of images, producing a different signal for each of the plurality of images; and
(c) the step of analyzing the signal comprises the step of analyzing each different signal produced for each of the plurality of images to determine if indicative spectral signals produced by the plurality of optical signaling components are present in the plurality of images, thereby establishing that the feature is associated with the object.

42. A method for probing an object with labeled probes to detect if any of a plurality of specific features is associated with the object, using an imaging system that does not spatially resolve locations of the labeled probes on any specific feature, the method comprising the steps of:
(a) for each specific feature to be detected, providing at least one labeled probe that selectively couples to a corresponding specific feature, wherein each labeled probe comprises a binding element that selectively binds to at least a portion of the specific feature, and at least one optical signaling component that is bound to the specific feature by the binding element;
(b) exposing said object to said at least one labeled probe for each specific feature to be detected, under conditions that cause each labeled probe to couple to at least a portion of its corresponding specific feature, if that corresponding specific feature is associated with said object, such that at least two optical signaling components become bound to each specific feature associated with said object, each of said at least two optical signaling components that is bound to each specific feature being uniquely optically discriminable based upon a multiplex of the light from the optical signaling components, without spatially resolving a location of each labeled probe coupled to a specific feature;
(c) simultaneously detecting light from all optical signaling components associated with said object, producing a corresponding signal; and
(d) analyzing the signal to detect each optical signaling component bound to any specific feature associated with the object, thereby determining which specific feature is associated with the object.

43. The method of claim 42, wherein the step of exposing said object to said at least one labeled probe comprises the step of exposing said object to a labeled probe having a plurality of optical signaling components, thereby binding the plurality of optical signaling components to said corresponding specific feature associated with the object.

44. The method of claim 43, wherein the step of exposing said object to a labeled probe comprises the step of exposing said object to a labeled probe that comprises a plurality of identical optical signaling components.

45. The method of claim 43, wherein the step of exposing said object to a labeled probe comprises the step of exposing said object to a labeled probe that comprises at least two different optical signaling components.

46. The method of claim 43, wherein the step of exposing said object to a labeled probe comprises the step of exposing said object to at least two labeled probes selected to selectively bind to different portions of a first specific feature, each of said at least two labeled probes comprising:
(a) a binding element that selectively binds to at least a portion of the first specific feature;
(b) at least one optical signaling component that is bound by the binding element to said at least a portion of the first specific feature, so that a plurality of optical signaling components are bound to the first specific feature.

47. The method of claim 42, wherein the step of simultaneously detecting light from all signaling components associated with said object comprising the steps of:
(a) collecting light from said object along a collection path, said light comprising a multiplexed optical signal from the optical components coupled to each feature;
(b) focusing the collected light to produce an image corresponding to the object; and
(c) detecting the image, said collected light forming the image including optical components indicative of the optical signal components that are bound to each specific feature associated with the object.

48. The method of claim 42, wherein the step of simultaneously detecting light from all optical signaling components bound to each feature associated with said object comprises the steps of:
(a) collecting light from said object along a collection path; and
(b) dispersing the light that is traveling along the collection path into a plurality of light beams, as a function of a plurality of different discriminable characteristics of the light;
(c) focusing each of the plurality of light beams to produce a respective image corresponding to that light beam, thereby generating a plurality of images; and
(d) detecting the plurality of images.

49. The method of claim 42, wherein each optical signaling component comprises a fluorescent dye, further comprising the step of directing sufficient energy toward said object, such that the fluorescent dye is excited to emit a fluorescent light comprising a uniquely discriminable characteristic of the optical signal component.

50. The method of claim 42, wherein an optical signature of said plurality of optical signaling components bound to each specific feature is uniquely discriminable based on an intensity of multiplexed light from the plurality of optical signal components.

51. The method of claim 42, wherein a spectral signature of the plurality of optical signaling components bound to a specific feature is uniquely discriminable based on its spectral composition of light from the plurality optical signal components.
